(19) United States
(12) Patent Application Publication
(10) Pub. No.: US 2019/0337889 A1
(43) Pub. Date: Nov. 7, 2019

(54) COMPOSITIONS AND METHODS OF REGULATING CANCER RELATED DISORDERS AND DISEASES

(71) Applicant: ONCO THERAPIES LLC, Pasadena, CA (US)

(72) Inventors: Anshu VASHISHTHA, Irvine, CA (US); Malini VASHISHTHA, Irvine, CA (US)

(21) Appl. No.: 15/769,039

(22) PCT Filed: Oct. 17, 2016
(86) PCT No.: PCT/US2016/057377

§ 371 Date: Apr. 17, 2018

(51) Int. Cl. C07C 233/43; C07C 211/27; C07C 211/30; C07C 215/32; C07C 215/34; C07C 215/52; C07C 215/60; C07C 215/76; C07C 215/86; C07C 217/94

(52) U.S. Cl. CPC C07C 233/43; C07C 211/27; C07C 211/30; C07C 215/32; C07C 215/34; A61K 45/06; C07C 215/60; C07C 215/76; C07C 215/86; C07C 217/94; C07C 215/52

(57) ABSTRACT

Provided herein are naphthylic derivative compounds, or pharmaceutically acceptable salts thereof, that are useful for inhibiting cancers. Also provided herein are methods of using effective amounts of said compounds, optionally with pharmaceutical carriers, for the treatment of cancers within human subjects.

### CROSS-REFERENCE

This application claims the benefit of U.S. Provisional Application No. 62/243,073, filed Oct. 18, 2015, which application is incorporated herein by reference.

### BACKGROUND OF THE INVENTION

The present filing relates to the composition and the use of certain classes of compounds including but not limited to, naphthalene derivatives or similar compounds for treatment of a disorder or disease, such as a glioblastoma, hepatocellular carcinoma, liver cancer, colon cancer, brain cancer and/or lung cancer, or other tumor, associated with susceptibility to successful treatment with such compounds, that may be related in some instances to a) altered beta adrenergic receptor activity b) altered cannabinoid receptor activity or c) altered serotonin receptor activity or other GPCR coupled receptor activity, or another method of identifying such susceptibility.

### SUMMARY OF THE INVENTION

Disclosed herein is a compound for treating or preventing a cancer comprising administering a compound having the structure of Formula I:

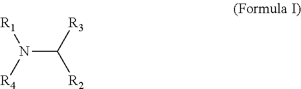

wherein, R₁ and R₃ are either hydrogen or alkyl groups; R₄ is hydrogen, substituted or unsubstituted alkyl, substituted or unsubstituted aryl, or substituted or unsubstituted alkyl chains terminating in a substituted or unsubstituted aryl; R₂ is a substituted or unsubstituted naphthyl group optionally linked by a substituted or unsubstituted alkyl chain;

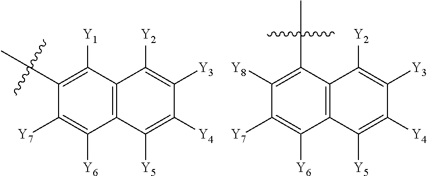

wherein, each Y₁-Y₈ are independently selected from hydrogen, deuterium, halogen (F, Cl, Br, I), hydroxyl, alkoxy, nitrogenous substituents such as primary amines, substituted secondary and tertiary amines, sulfurous substituents including SH, sulfoxides, sulfones, sulfonamides, substituted or unsubstituted alkyl and substituted or unsubstituted aryls.

In some embodiments, R₁ and R₃ are either hydrogen or alkyl groups; R₄ is hydrogen, substituted or unsubstituted alkyl, substituted or unsubstituted aryl, or substituted or unsubstituted alkyl chains terminating in a substituted or unsubstituted aryls; R₂ is a substituted or unsubstituted naphthyl group optionally linked by a substituted or unsubstituted alkyl chain; and each Y₁-Y₈ are independently selected from hydrogen, deuterium, halogen (F, Cl, Br, I), hydroxyl, alkoxy, primary amines, substituted secondary and tertiary amines, thiols, sulfoxides, sulfones, sulfanamides, substituted or unsubstituted alkyl and substituted or unsubstituted aryls.

In some embodiments, R₁ and R₃ are either hydrogen or alkyl groups; R₄ is hydrogen, a substituted or unsubstituted aryl or substituted or unsubstituted alkyl chains terminating in a substituted or unsubstituted aryls; R₂ is a substituted or unsubstituted naphthyl group optionally linked by a substituted or unsubstituted alkyl chain; and each Y₁-Y₈ are independently selected from hydrogen, deuterium, halogen (F, Cl, Br, I), hydroxyl, alkoxy, primary amines, substituted secondary and tertiary amines, thiols, sulfoxides, sulfones, sulfanamides, substituted or unsubstituted alkyl and substituted or unsubstituted aryls.

In some embodiments, R₁ and R₃ are either hydrogen or alkyl groups; R₄ is hydrogen or a substituted or unsubstituted alkyl chains terminating in a substituted or unsubstituted aryls;

R₂ is a substituted or unsubstituted naphthyl group optionally linked by a substituted or unsubstituted alkyl chain; and each Y₁-Y₈ are independently selected from hydrogen, deuterium, halogen (F, Cl, Br, I), hydroxyl, alkoxy, primary amines, substituted secondary and tertiary amines, thiols, sulfoxides, sulfones, sulfanamides, substituted or unsubstituted alkyl and substituted or unsubstituted aryls.

In some embodiments, R₁ and R₃ are either hydrogen or alkyl groups; R₄ is hydrogen or an alkyl chain terminating in a hydroxy substituted aryls; R₂ is a substituted or unsubstituted naphthyl group optionally linked by a substituted or unsubstituted alkyl chain; and each Y₁-Y₈ are independently selected from hydrogen, deuterium, halogen (F, Cl, Br, I), hydroxyl, alkoxy, primary amines, substituted secondary and tertiary amines, thiols, sulfoxides, sulfones, sulfanamides, substituted or unsubstituted alkyl and substituted or unsubstituted aryls.

In some embodiments, R₁ and R₃ are either hydrogen or alkyl groups; R₄ is hydrogen or an alkyl chain terminating in a hydroxy substituted aryls; R₂ is a substituted or unsubstituted naphthyl group optionally linked by a substituted or unsubstituted alkyl chain; and each Y₁-Y₈ are independently selected from hydrogen, hydroxyl, and alkoxy substituents.

In some embodiments, the compound is selected from the group consisting of: 1-(naphthalen-1-yl)propan-2-amine; 1-naphthalen-1-yl)ethan-1-amine; 1-(naphthalen-2-yl)ethan-1-amine; 1-(6-methoxynaphthalen-2-yl)ethan-1-amine; 1-(1-amino-2-methylpropyl)naphthalen-2-ol; 1-(naphthalen-2-yl)propan-2-amine; 4-(2-((1-(naphthalen-1-yl)propan-2-yl)amino)ethyl)phenol; 4-((1-(naphthalen-1-yl)propan-2-yl)amino)phenol; 4-((1-(naphthalen-2-yl)propan-2-yl)amino)phenol; 4-(1-hydroxy-2-((1-(naphthalen-1-yl)propan-2-yl)amino)ethyl)phenol; 2-((1-(naphthalen-2-yl)propan-2-yl)amino)-1-phenylethan-1-ol; 4-(2-((1-(naphthalen-2-yl)propan-2-yl)amino)ethyl)phenol; 3-(1-hydroxy-2-((1-(naphthalen-1-yl)propan-2-yl)amino)ethyl)phenol; 3-(1-hydroxy-2-((1-(naphthalen-2-yl)propan-2-yl)amino)ethyl)phenol; 4-(1-hydroxy-2-((1-(naphthalen-2-yl)propan-2-yl)amino)ethyl)benzene-1,2-diol; 4-(1-hydroxy-2-((1-(naphthalen-1-yl)propan-2-yl)amino)ethyl)benzene-1,2-diol; 4-(1-hydroxy-2-((1-(naphthalen-2-yl)propan-2-yl)amino)ethyl)phenol; 4-(1-hydroxy-2-((1-(naphthalen-1-yl)propan-2-yl)amino)ethyl)phenol; 2-(1-hydroxy-2-((1-(naphthalen-1-yl)propan-2-yl)amino)ethyl)phenol; 2-(1-hydroxy-2-((1-(naphthalen-2-yl)propan-2-yl)amino)ethyl)phenol; 4-(2-((1-(4-methylnaphthalen-1-yl)propan-2-yl)amino)ethyl)phenol; 4-(2-((1-(5-methylnaphthalen-1-yl)propan-2-yl)amino)ethyl)phenol; 4-(1-hydroxy-2-((1-(4-methylnaphthalen-1-yl)propan-2-yl)amino)ethyl)benzene-1,2-diol; 3-(1-hydroxy-2-((1-(4-methylnaphthalen-1-yl)propan-2-yl)amino)ethyl)phenol; 2-((1-(4-methylnaphthalen-1-yl)propan-2-yl)amino)-1-phenylethan-1-ol; 4-(1-hydroxy-2-((1-(4-methylnaphthalen-1-yl)propan-2-yl)amino)ethyl)phenol; 4-(((1-(4-methylnaphthalen-1-yl)propan-2-yl)amino)methyl)phenol; 4-(((1-(naphthalen-1-yl)propan-2-yl)amino)methyl)phenol and 4-(((1-(naphthalen-2-yl)propan-2-yl)amino)methyl)phenol.

In some embodiments, the compound is 4-(2-((1-(naphthalen-1-yl)propan-2-yl)amino)ethyl)phenol.

Disclosed herein are methods of treating cancer using a compound of Formula I.

Also disclosed herein are methods of administering a therapeutically effective amount of the compound of Formula I.

In some embodiments, the compound used is selected from the group of compounds: 1-(naphthalen-1-yl)propan-2-amine; 1-naphthalen-1-yl)ethan-1-amine; 1-(naphthalen-2-yl)ethan-1-amine; 1-(6-methoxynaphthalen-2-yl)ethan-1-amine; 1-(1-amino-2-methylpropyl)naphthalen-2-ol; 1-(naphthalen-2-yl)propan-2-amine; 4-(2-((1-(naphthalen-1-yl)propan-2-yl)amino)ethyl)phenol; 4-((1-(naphthalen-1-yl)propan-2-yl)amino)phenol; 4-((1-(naphthalen-2-yl)propan-2-yl)amino)phenol; 4-(1-hydroxy-2-((1-(naphthalen-1-yl)propan-2-yl)amino)ethyl)phenol; 2-((1-(naphthalen-2-yl)propan-2-yl)amino)-1-phenylethan-1-ol; 4-(2-((1-(naphthalen-2-yl)propan-2-yl)amino)ethyl)phenol; 3-(1-hydroxy-2-((1-(naphthalen-1-yl)propan-2-yl)amino)ethyl)phenol; 3-(1-hydroxy-2-((1-(naphthalen-2-yl)propan-2-yl)amino)ethyl)phenol; 4-(1-hydroxy-2-((1-(naphthalen-2-yl)propan-2-yl)amino)ethyl)benzene-1,2-diol; 4-(1-hydroxy-2-((1-(naphthalen-1-yl)propan-2-yl)amino)ethyl)benzene-1,2-diol; 4-(1-hydroxy-2-((1-(naphthalen-2-yl)propan-2-yl)amino)ethyl)phenol; 4-(1-hydroxy-2-((1-(naphthalen-1-yl)propan-2-yl)amino)ethyl)phenol; 2-(1-hydroxy-2-((1-(naphthalen-1-yl)propan-2-yl)amino)ethyl)phenol; 2-(1-hydroxy-2-((1-(naphthalen-2-yl)propan-2-yl)amino)ethyl)phenol; 4-(2-((1-(4-methylnaphthalen-1-yl)propan-2-yl)amino)ethyl)phenol; 4-(2-((1-(5-methylnaphthalen-1-yl)propan-2-yl)amino)ethyl)phenol; 4-(1-hydroxy-2-((1-(4-methylnaphthalen-1-yl)propan-2-yl)amino)ethyl)benzene-1,2-diol; 3-(1-hydroxy-2-((1-(4-methylnaphthalen-1-yl)propan-2-yl)amino)ethyl)phenol; 2-((1-(4-methylnaphthalen-1-yl)propan-2-yl)amino)-1-phenylethan-1-ol; 4-(1-hydroxy-2-((1-(4-methylnaphthalen-1-yl)propan-2-yl)amino)ethyl)phenol; 4-(((1-(4-methylnaphthalen-1-yl)propan-2-yl)amino)methyl)phenol; 4-(((1-(naphthalen-1-yl)propan-2-yl)amino)methyl)phenol and 4-(((1-(naphthalen-2-yl)propan-2-yl)amino)methyl)phenol.

In some embodiments, the compound used is selected from the group of compounds: 1-(naphthalen-2-yl)propan-2-amine and 4-(2-((1-(naphthalen-1-yl)propan-2-yl)amino)ethyl)phenol.

Also disclosed herein are methods of administering a therapeutically effective amount of a compound selected from the group consisting of 1-(naphthalen-1-yl)propan-2-amine; 1-naphthalen-1-yl)ethan-1-amine; 1-(naphthalen-2-yl)ethan-1-amine; 1-(6-methoxynaphthalen-2-yl)ethan-1-amine; 1-(1-amino-2-methylpropyl)naphthalen-2-ol; 1-(naphthalen-2-yl)propan-2-amine; 4-(2-((1-(naphthalen-1-yl)propan-2-yl)amino)ethyl)phenol; 4-((1-(naphthalen-1-yl)propan-2-yl)amino)phenol; 4-((1-(naphthalen-2-yl)propan-2-yl)amino)phenol; 4-(1-hydroxy-2-((1-(naphthalen-1-yl)propan-2-yl)amino)ethyl)phenol; 2-((1-(naphthalen-2-yl)propan-2-yl)amino)-1-phenylethan-1-ol; 4-(2-((1-(naphthalen-2-yl)propan-2-yl)amino)ethyl)phenol; 3-(1-hydroxy-2-((1-(naphthalen-1-yl)propan-2-yl)amino)ethyl)phenol; 3-(1-hydroxy-2-((1-(naphthalen-2-yl)propan-2-yl)amino)ethyl)phenol; 4-(1-hydroxy-2-((1-(naphthalen-2-yl)propan-2-yl)amino)ethyl)benzene-1,2-diol; 4-(1-hydroxy-2-((1-(naphthalen-1-yl)propan-2-yl)amino)ethyl)benzene-1,2-diol; 4-(1-hydroxy-2-((1-(naphthalen-2-yl)propan-2-yl)amino)ethyl)phenol; 4-(1-hydroxy-2-((1-(naphthalen-1-yl)propan-2-yl)amino)ethyl)phenol; 2-(1-hydroxy-2-((1-(naphthalen-1-yl)propan-2-yl)amino)ethyl)phenol; 2-(1-hydroxy-2-((1-(naphthalen-2-yl)propan-2-yl)amino)ethyl)phenol; 4-(2-((1-(4-methylnaphthalen-1-yl)propan-2-yl)amino)ethyl)phenol; 4-(2-((1-(5-methylnaphthalen-1-yl)propan-2-yl)amino)ethyl)phenol; 4-(1-hydroxy-2-((1-(4-methylnaphthalen-1-yl)propan-2-yl)amino)ethyl)benzene-1,2-diol; 3-(1-hydroxy-2-((1-(4-methylnaphthalen-1-yl)propan-2-yl)amino)ethyl)phenol; 2-((1-(4-methylnaphthalen-1-yl)propan-2-yl)amino)-1-phenylethan-1-ol; 4-(1-hydroxy-2-((1-(4-methylnaphthalen-1-yl)propan-2-yl)amino)ethyl)phenol; 4-(((1-(4-methylnaphthalen-1-yl)propan-2-yl)amino)methyl)phenol; 4-(((1-(naphthalen-1-yl)propan-2-yl)amino)methyl)phenol and 4-(((1-(naphthalen-2-yl)propan-2-yl)amino)methyl)phenol.

Disclosed herein are methods of administering a therapeutically effective amount of 1-(naphthalen-2-yl)propan-2-amine and 4-(2-((1-(naphthalen-1-yl)propan-2-yl)amino)ethyl)phenol.

Disclosed herein also are methods of administering a therapeutically effective amount of a compound selected from the group consisting of: 1-(naphthalen-1-yl)propan-2-amine; 1-naphthalen-1-yl)ethan-1-amine; 1-(naphthalen-2-yl)ethan-1-amine; 1-(6-methoxynaphthalen-2-yl)ethan-1-amine; 1-(1-amino-2-methylpropyl)naphthalen-2-ol; 1-(naphthalen-2-yl)propan-2-amine; 4-(2-((1-(naphthalen-1-yl)propan-2-yl)amino)ethyl)phenol; 4-((1-(naphthalen-1-yl)propan-2-yl)amino)phenol; 4-((1-(naphthalen-2-yl)propan-2-yl)amino)phenol; 4-(1-hydroxy-2-((1-(naphthalen-1-yl)propan-2-yl)amino)ethyl)phenol; 2-((1-(naphthalen-2-yl)propan-2-yl)amino)-1-phenylethan-1-ol; 4-(2-((1-(naphthalen-2-yl)propan-2-yl)amino)ethyl)phenol; 3-(1-hydroxy-2-((1-(naphthalen-1-yl)propan-2-yl)amino)ethyl)phenol; 3-(1-hydroxy-2-((1-(naphthalen-2-yl)propan-2-yl)amino)ethyl)phenol; 4-(1-hydroxy-2-((1-(naphthalen-2-yl)propan-2-yl)amino)ethyl)benzene-1,2-diol; 4-(1-hydroxy-2-((1-(naphthalen-1-yl)propan-2-yl)amino)ethyl)benzene-1,2-diol; 4-(1-hydroxy-2-((1-(naphthalen-2-yl)propan-2-yl)amino)ethyl)phenol; 4-(1-hydroxy-2-((1-(naphthalen-1-yl)propan-2-yl)amino)ethyl)phenol; 2-(1-hydroxy-2-((1-(naphthalen-1-yl)propan-2-yl)amino)ethyl)phenol; 2-(1-hydroxy-2-((1-(naphthalen-2-yl)propan-2-yl)amino)ethyl)phenol; 4-(2-((1-(4-methylnaphthalen-1-yl)propan-2-yl)amino)ethyl)phenol; 4-(2-((1-(5-methylnaphthalen-1-yl)propan-2-yl)amino)ethyl)phenol; 4-(1-hydroxy-2-((1-(4-methylnaphthalen-1-yl)propan-2-yl)amino)ethyl)benzene-1,2-diol; 3-(1-hydroxy-2-((1-(4-methylnaphthalen-1-yl)propan-2-yl)amino)ethyl)phenol; 2-((1-(4-methylnaphthalen-1-yl)propan-2-yl)amino)-1-phenylethan-1-ol; 4-(1-hydroxy-2-((1-(4-methylnaphthalen-1-yl)propan-2-yl)amino)ethyl)phenol; 4-(((1-(4-methylnaphthalen-1-yl)propan-2-yl)amino)methyl)phenol; 4-(((1-(naphthalen-1-yl)propan-2-yl)amino)methyl)phenol and 4-(((1-(naphthalen-2-yl)propan-2-yl)amino)methyl)phenol or a combination thereof.

Disclosed herein are methods of administering a therapeutically effective amount of 1-(naphthalen-2-yl)propan-2-amine or 4-(2-((1-(naphthalen-1-yl)propan-2-yl)amino)ethyl)phenol or a combination thereof.

In some embodiments, the disorder or disease is liver cancer, brain cancer, lung cancer, breast cancer or any cancer that responds to these compounds.

In some embodiments, the disorder or disease is liver cancer, brain cancer, lung cancer or breast cancer.

In some embodiments, the disorder or disease is liver cancer.

In some embodiments, the disorder or disease is brain cancer.

In some embodiments, the disorder or disease is lung cancer.

In some embodiments, the disorder or disease is breast cancer.

The methods described herein can be applied, in some embodiments, to cases where inhibiting one or more signs or symptoms associated with the disease or disorder comprises inhibiting cellular growth, such as tumor or cancer cell growth (or both), tumor volume, or a combination thereof.

The methods described herein can also be applied, in some embodiments, to cases further comprising administering an additional therapeutic agent, such as prior to, concurrent with, or subsequent to administering a compound.

In some embodiments, this includes cases wherein the additional therapeutic agent is a chemotherapeutic agent or agent with antitumor activity.

In some embodiments, this includes cases wherein administering a therapeutically effective amount of a compound is done so with use of a pharmaceutically acceptable carrier.

In some embodiments this also includes cases wherein the subject is a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A: The result of cytotoxicity assay comparing compounds NT001 through NT015 are shown. The results are plotted as percent inhibition of cell viability measured by XTT assay. The results on HepG2 cells are shown. Compounds with an effect on viability up to 200 μM are shown.

FIG. 1B: The result of cytotoxicity assay comparing compounds NT001 through NT015 are shown. The results are plotted as percent inhibition of cell viability measured by XTT assay. The results on 1321N1 cells are shown. Compounds with an effect on viability up to 200 μM are shown.

FIG. 2: The results of XTT assay after treatments with compounds NT015 to NT037 on 1321N1 cells, HepG2 cells and PC3 cells are shown. The compounds were tested at concentrations of 0.01 μM, 10 μM, 100 μM and 200 μM. The control cells were treated with 1:100 DMSO. The y-axis shows the level of absorbance at 490 nM. The outer and inner refers to inner and outer wells with medium and no cells to which XIT reagent was added. (n=4)

FIG. 3: Caspase assay was done on HepG2 cells 48 hours after addition of compounds NT015, NT019, NT020, NT022, and NT027. The fluorescence ratio to untreated cells was plotted on the y-axis. The compounds NT015, NT019, NT022, NT027 showed an increase in caspase activity at a 100 uM concentration. The control compound ionomycin did not show an increase in caspase activation at 10 uM concentration. (n=4)

FIG. 4: The area of A549 xenograft implant was measured after implantation and 4 days after implantation with or without treatment with selected compounds (NT015, NT016, NT026, NT027, NT029, and NT039) at a concentration of 100 uM. The compounds NT016, NT026, NT027, NT029 and NT039 showed efficacy of reducing the area of xenograft significantly. (n=10 to 15)

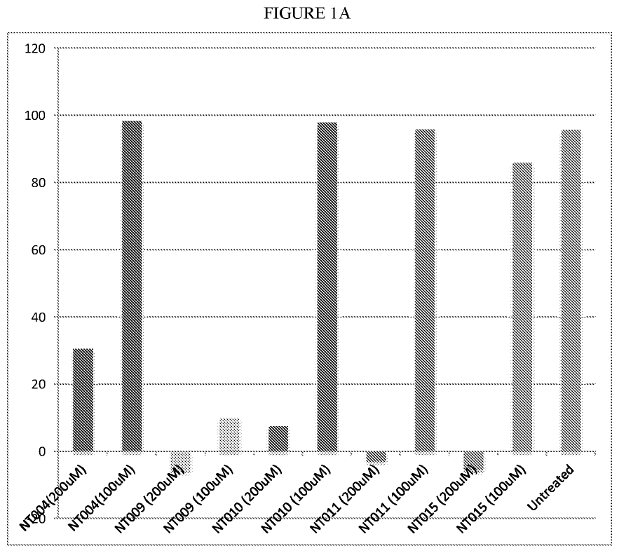

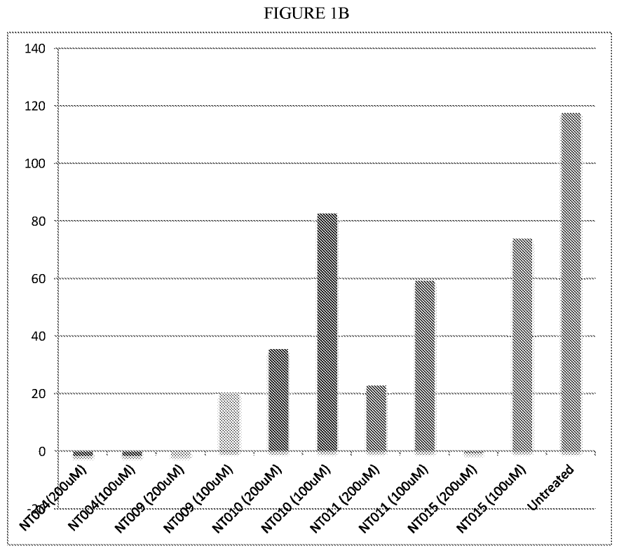

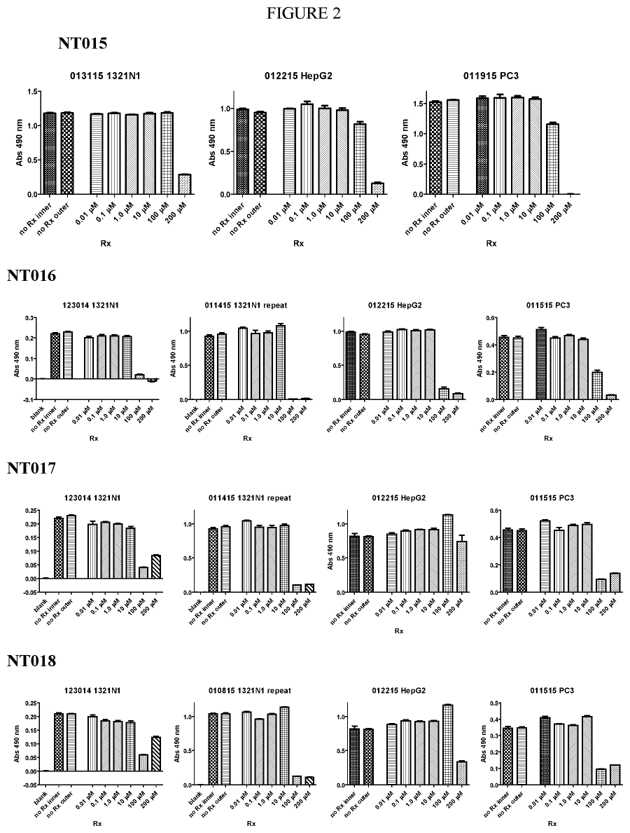

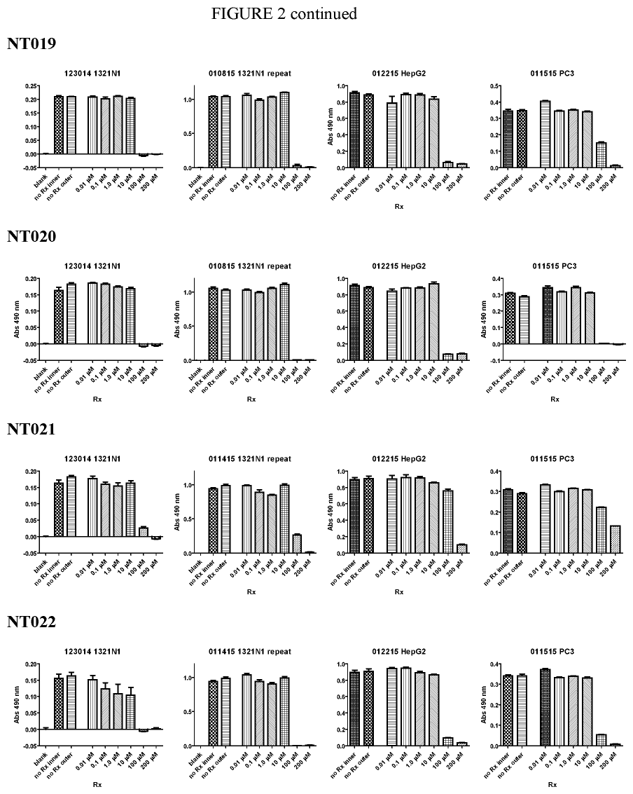

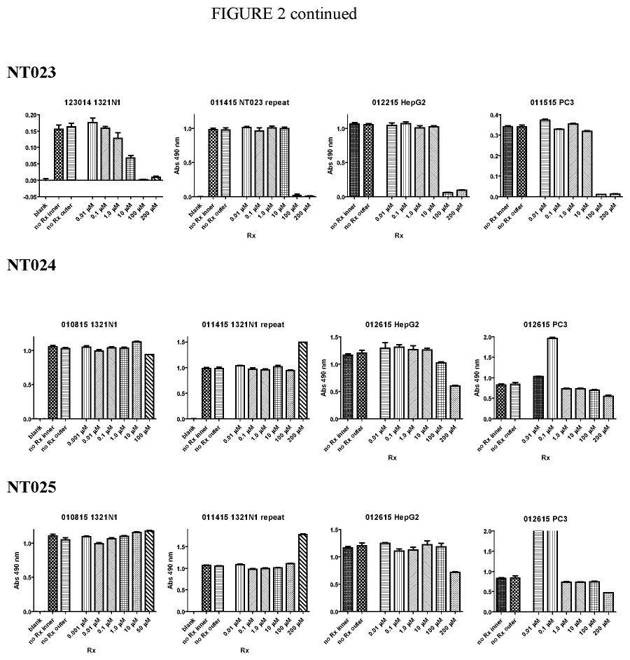

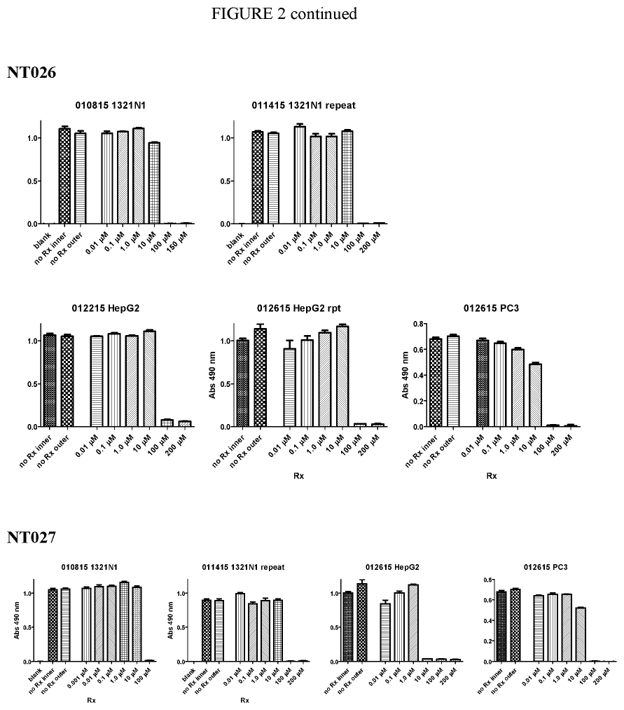

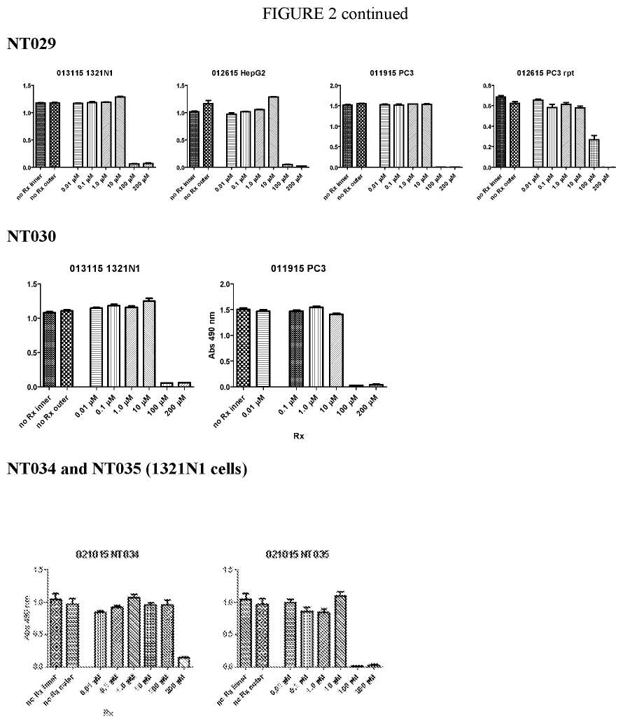

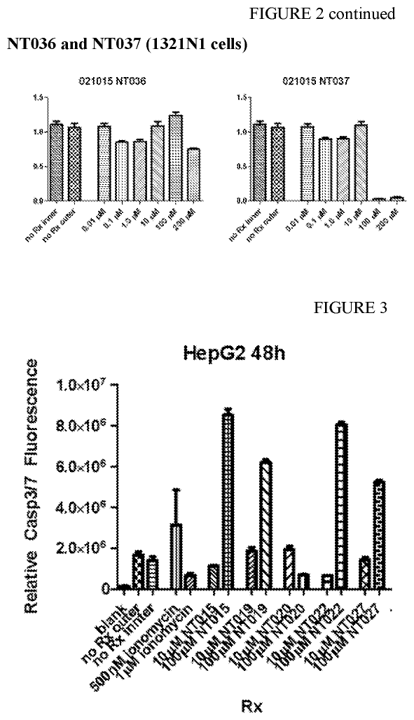

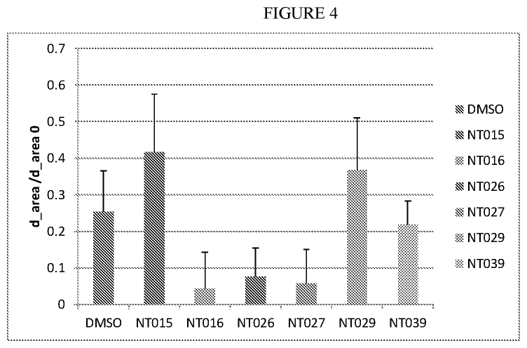

### DETAILED DESCRIPTION OF THE INVENTION

Disclosed herein is the finding that specific compounds described herein, such as 1-(naphthalen-2-yl)propan-2-amine and 4-(2-((1-(naphthalen-1-yl)propan-2-yl)amino)ethyl)phenol, inhibit the growth of various types of tumor cells, including astrocytoma tumor cells, glioblastoma tumor cells, medulloblastoma tumor cells, hepatocellular carcinoma cells, and lung cancer cells. The compounds were observed to inhibit the growth of human-derived hepatocellular carcinoma cells (HepG2) and human-derived glioblastoma and human-derived astrocytoma cells and lung cancer cells using in vitro incubation and in vivo xenograft assays in zebrafish.

Unless otherwise explained, all technical and scientific terms used herein have the same meaning as commonly understood by one of ordinary skill in the art to which the disclosed subject matter belongs. Definitions of common terms in chemistry may be found in The McGraw-Hill Dictionary of Chemical Terms, 1985, and The Condensed Chemical Dictionary, 1981.

Except as otherwise noted, any quantitative values are approximate whether the word “about” or “approximately” or the like are stated or not. The materials, methods, and examples described herein are illustrative only and not intended to be limiting. Any molecular weight or molecular mass values are approximate and are provided only for description. Except as otherwise noted, the methods and techniques of the present invention are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978.

### Definition of Terms

In order to facilitate review of the various embodiments disclosed herein, the following explanations of specific terms are provided:

Acyl: A group of the formula RC(O)— wherein R is an organic group.

Aryl: “Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Bickel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, hydroxy, alkoxy optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)R^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

Acyloxy: A group having the structure —OC(O)R, where R may be an optionally substituted alkyl or optionally substituted aryl. “Lower acyloxy” groups are those where R contains from 1 to 10 (such as from 1 to 6) carbon atoms.

Administration: To provide or give a subject a composition, such as a pharmaceutical composition including one or more compounds by any effective route. Exemplary routes of administration include, but are not limited to, injection (such as subcutaneous, intramuscular, intradermal, intraperitoneal (IP), and intravenous (IV)), oral, sublingual, rectal, transdermal, intranasal, vaginal and inhalation routes.

Alkoxy: A radical (or substituent) having the structure —O—R, where R is a substituted or unsubstituted alkyl. Methoxy (—OCH3) is an exemplary alkoxy group. In a substituted alkoxy, R is alkyl substituted with a non-interfering substituent. “Thioalkoxy” refers to —S—R, where R is substituted or unsubstituted alkyl. “Haloalkyloxy” means a radical —OR where R is a haloalkyl.

Alkoxy carbonyl: A group of the formula —C(O)OR, where R may be an optionally substituted alkyl or optionally substituted aryl. “Lower alkoxy carbonyl” groups are those where R contains from 1 to 10 (such as from 1 to 6) carbon atoms.

Alkyl: An acyclic, saturated, branched- or straight-chain hydrocarbon radical, which, unless expressly stated otherwise, contains from one to fifteen carbon atoms; for example, from one to ten, from one to six, or from one to four carbon atoms. This term includes, for example, groups such as methyl, ethyl, n-propyl, isopropyl, isobutyl, t-butyl, pentyl, heptyl, octyl, nonyl, decyl, or dodecyl. The term “lower alkyl” refers to an alkyl group containing from one to ten carbon atoms. Unless expressly referred to as an “unsubstituted alkyl,” alkyl groups can either be unsubstituted or substituted. An alkyl group can be substituted with one or more substituents (for example, up to two substituents for each methylene carbon in an alkyl chain) independently selected from alkyl, alkenyl, alkynyl, halo, fluoroalkyl, oxo, thioxo, cyano, nitro, hydroxyl, amino, alkoxy, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl or optionally substituted heteroarylalkyl.

Alkylene: Refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

Alkynylene: Refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atom (e.g., C₂ alkylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —(O)OR^{a}, —(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

Amino carbonyl (carbamoyl): A group of the formula C(O)N(R)R′, wherein R and R′ are independently of each other hydrogen or a lower alkyl group.

Astrocytoma: A tumor of the brain that originates in astrocytes. An astrocytoma is an example of a primary tumor. Astrocytomas are the most common glioma, and can occur in most parts of the brain and occasionally in the spinal cord. However, astrocytomas are most commonly found in the cerebrum. In one example, an astrocytoma is inhibited by administering to a subject a therapeutic effective amount of NT015, NT016 or a combination thereof, thereby inhibiting astrocytoma growth.

β2-adrenergic receptor: (β2-AR): A subtype of adrenergic receptors that are members of the G-protein coupled receptor family. β2-AR subtype is involved in respiratory diseases, cardiovascular diseases, premature labor and, as disclosed herein, tumor development. Increased expression of β2-ARs can serve as therapeutic targets. Currently, a number of drugs e.g., albuterol, formoterol, isoproterenol, or salmeterol have β2-AR agonist activities. As disclosed herein, the compounds described herein may be β2-AR agonists.

Blood-brain barrier (BBB): The barrier formed by epithelial cells in the capillaries that supply the brain and central nervous system. This barrier selectively allows entry of substances such as water, oxygen, carbon dioxide, and nonionic solutes such as glucose, alcohol, and general anesthetics, while blocking entry of other substances. Some small molecules, such as amino acids, are taken across the barrier by specific transport mechanisms.

Cannabinoid Receptors: A class of cell membrane receptors under the G protein-coupled receptor superfamily. The cannabinoid receptors contain seven transmembrane spanning domains. Cannabinoid receptors are activated by three major groups of ligands, endocannabinoids (produced by the mammalian body), plant cannabinoids (such as THC, produced by the cannabis plant) and synthetic cannabinoids (such as HU-210). All of the endocannabinoids and plant cannabinoids are lipophilic, i.e. fat soluble, compounds. Two subtypes of cannabinoid receptors are CB1 (see GenBank Accession No. NM_033181 mRNA and UniProt P21554, each of which is hereby incorporated by reference as of May 23, 2012) and CB2 (see GenBank Accession No. NM_001841 mRNA and UniProt P34972, each of which is hereby incorporated by reference as of May 23, 2012). The CB1 receptor is expressed mainly in the brain (central nervous system, CNS), but also in the lungs, liver and kidneys. The CB2 receptor is expressed mainly in the immune system and in hematopoietic cells. Additional non-CB1 and non-CB2 include GPR55 (GenBank Accession No. NM_005683.3 or NP_005674.2 protein, each of which is hereby incorporated by reference as of May 23, 2012), GPR119 (GenBank Accession No. NM_178471.2 or NP 848566.1 protein, each of which is hereby incorporated by reference as of May 23, 2012) and GPR18 (also known as N-arachidonyl glycine receptor and involved in microglial migration, GenBank Accession No. NM_001098200 mRNA, NP_001091670.1, each of which is hereby incorporated by reference as of May 23, 2012).

Carbamate: A group of the formula —OC(O)N(R)—, wherein R is H, or an aliphatic group, such as a lower alkyl group or an aralkyl group.

Carbocyclyl: The term carbocyclyl refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C-C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}, —C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

Chemotherapy; chemotherapeutic agents: As used herein, any chemical agent with therapeutic usefulness in the treatment of diseases characterized by abnormal cell growth. Such diseases include tumors, neoplasms, and cancer as well as diseases characterized by hyperplastic growth. In one embodiment, a chemotherapeutic agent is an agent of use in treating neoplasms such as solid tumors, including a tumor associated with susceptible receptor activity and/or expression. In one embodiment, a chemotherapeutic agent is radioactive molecule. In some embodiments, a susceptible receptor regulator, such as NT015, NT016 or a combination thereof is a chemotherapeutic agent. In one example, a chemotherapeutic agent is carmustine, lomustine, procarbazine, streptozocin, or a combination thereof. One of skill in the art can readily identify a chemotherapeutic agent of use (e.g., see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L., Berkery R. (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

Control or Reference Value: A “control” refers to a sample or standard used for comparison with a test sample. In some embodiments, the control is a historical control or standard reference value or range of values (such as a previously tested control sample, such as a group of subjects which do not have a tumor expressing susceptible receptor or group of samples that represent baseline or normal values, such as the level of susceptible receptor in tumor tissue that does not respond to treatment with NT-015, NT016, or a combination thereof).

Derivative: A chemical substance that differs from another chemical substance by one or more functional groups. Preferably, a derivative (such as a naphthalene derivative) retains a biological activity (CB receptor activation) of a molecule from which it was derived (such as a naphthalene derivative capable of regulating a CB receptor, such as GPR55).

Effective amount: An amount of agent that is sufficient to generate a desired response, such as reducing or inhibiting one or more signs or symptoms associated with a condition or disease. When administered to a subject, a dosage will generally be used that will achieve target tissue concentrations. In some examples, an “effective amount” is one that treats one or more symptoms and/or underlying causes of any of a disorder or disease. In some examples, an “effective amount” is a “therapeutically effective amount” in which the agent alone with an additional therapeutic agent(s) (for example a chemotherapeutic agent) induces the desired response such as treatment of a tumor. In one example, a desired response is to decrease tumor size or metastasis in a subject to whom the therapy is administered. Tumor metastasis does not need to be completely eliminated for the composition to be effective. For example, a composition can decrease metastasis by a desired amount, for example by at least 20%, at least 50%, at least 60%, at least 70%, at least 80%, at least 90%, at least 95%, at least 98%, or even at least 100% (elimination of the tumor), as compared to metastasis in the absence of the composition.

In particular examples, it is an amount of an agent effective to decrease a number of carcinoma cells, such as in a subject to whom it is administered, for example a subject having one or more carcinomas. The cancer cells do not need to be completely eliminated for the composition to be effective. For example, a composition can decrease the number of cancer cells by a desired amount, for example by at least 20%, at least 50%, at least 60%, at least 70%, at least 80%, at least 90%, at least 95%, at least 98%, or even at least 100% (elimination of detectable cancer cells), as compared to the number of cancer cells in the absence of the composition.

In some examples, an effective amount is the amount of NT015 or NT016 useful in reducing, inhibiting, and/or treating a disorder or disease associated with expression its receptor. Ideally, a therapeutically effective amount of an agent is an amount sufficient to reduce, inhibit, and/or treat the disorder in a subject without causing a substantial cytotoxic effect in the subject.

The effective amount of a composition useful for reducing, inhibiting, and/or treating a disorder in a subject will be dependent on the subject being treated, the severity of the disorder, and the manner of administration of the therapeutic composition. Effective amounts a therapeutic agent can be determined in many different ways, such as assaying for a reduction in tumor size or improvement of physiological condition of a subject having a tumor, such as a brain tumor. Effective amounts also can be determined through various in vitro, in vivo or in situ assays.

Glioblastoma: A common and malignant form of a primary brain tumor. A glioblastoma is a grade IV astrocytoma and usually spreads rapidly in the brain.

Heteroaryl: Refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b] [1,4]dioxepinyl, benzo[b] [1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a, 7,8,9,10,10a-octahydrobenzo [h] quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

Heterocyclyl: The term heterocyclyl refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

Isomers: Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that contain two or more chiral centers and are not mirror images of one another are termed “diastereomers.” Stereoisomers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−) isomers, respectively). A chiral compound can exist as either an individual enantiomer or as a mixture thereof. A mixture containing equal proportions of the enantiomers is called a “racemic mixture.”

The compounds described herein may possess one or more asymmetric centers; such compounds can therefore be produced as individual (R), (S), (R,R′), (R,S′)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well known in the art (see, e.g., March, Advanced Organic Chemistry, 4th edition, New York: John Wiley and Sons, 1992, Chapter 4).

Optional: “Optional” or “optionally” means that the subsequently described event or circumstance can but need not occur, and that the description includes instances where said event or circumstance occurs and instances where it does not.

Pharmaceutically Acceptable Carriers: The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds or molecules, such as one or more nucleic acid molecules, proteins or antibodies that bind these proteins, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phenyl: Phenyl groups may be unsubstituted or substituted with one, two or three substituents, with substituent(s) independently selected from alkyl, heteroalkyl, aliphatic, heteroaliphatic, thioalkoxy, halo, haloalkyl (such as —CF3), nitro, cyano, —OR (where R is hydrogen or alkyl), —N(R)R′ (where R and R′ are independently of each other hydrogen or alkyl), —COOR (where R is hydrogen or alkyl) or —C(O)N(R′)R″ (where R′ and R″ are independently selected from hydrogen or alkyl).

Purified: The term “purified” does not require absolute purity; rather, it is intended as a relative term. Thus, for example, a purified preparation is one in which a desired component such as an (R,R′)-enantiomer of NT016 is more enriched than it was in a preceding environment such as in a (±)-NT016ener mixture. A desired component such as (R,R′)-enantiomer of cancer killer is considered to be purified, for example, when at least about 70%, 80%, 85%, 90%, 92%, 95%, 97%, 98%, or 99% of a sample by weight is composed of the desired component. Purity of a compound may be determined, for example, by high performance liquid chromatography (HPLC) or other conventional methods. In an example, the specific cancer killer enantiomers are purified to represent greater than 90%, often greater than 95% of the other enantiomers present in a purified preparation. In other cases, the purified preparation may be essentially homogeneous, wherein other stereoisomers are less than 1%.

Compounds described herein may be obtained in a purified form or purified by any of the means known in the art, including silica gel and/or alumina chromatography. See, e.g., Introduction to Modern Liquid Chromatography, 2nd Edition, ed. by Snyder and Kirkland, New York: John Wiley and Sons, 1979; and Thin Layer Chromatography, ed. by Stahl, New York: Springer Verlag, 1969. In an example, a compound includes purified cancer killer with a purity of at least about 70%, 80%, 85%, 90%, 92%, 95%, 97%, 98%, or 99% of a sample by weight relative to other contaminants. In a further example, a compound includes at least two purified stereoisomers each with a purity of at least about 70%, 80%, 85%, 90%, 92%, 95%, 97%, 98%, or 99% of a sample by weight relative to other contaminants. For instance, a compound can include a substantially purified (R,R′)-compound and a substantially purified (R,S′)-compound.

Subject: The term “subject” includes both human and veterinary subjects, for example, humans, non-human primates, dogs, cats, horses, rats, mice, and cows. Similarly, the term mammal includes both human and non-human mammals.

Solvate: The term “solvate” means a physical association of a compound with one or more solvent molecules. This physical association involves varying degrees of ionic and covalent bonding, including by way of example covalent adducts and hydrogen bonded solvates. In certain instances the solvate will be capable of isolation, for example when one or more solvent molecules are incorporated in the crystal lattice of the crystalline solid. “Solvate” encompasses both solution-phase and isolable solvates. Representative solvates include ethanol-associated compound, methanol-associated compounds, and the like. “Hydrate” is a solvate wherein the solvent molecule(s) is/are H₂O.

The disclosed compounds also encompass salts including, if several salt-forming groups are present, mixed salts and/or internal salts. The salts are generally pharmaceutically acceptable salts that are non-toxic. Salts may be of any type (both organic and inorganic), such as fumarates, hydrobromides, hydrochlorides, sulfates and phosphates. In an example, salts include non-metals (e.g., halogens) that form group VII in the periodic table of elements. For example, compounds may be provided as a hydrobromide salt.

Additional examples of salt-forming groups include, but are not limited to, a carboxyl group, a phosphonic acid group or a boronic acid group, that can form salts with suitable bases. These salts can include, for example, nontoxic metal cations, which are derived from metals of groups IA, IB, IIA and IIB of the periodic table of the elements. In one embodiment, alkali metal cations such as lithium, sodium or potassium ions, or alkaline earth metal cations such as magnesium or calcium ions can be used. The salt can also be a zinc or an ammonium cation. The salt can also be formed with suitable organic amines, such as unsubstituted or hydroxyl-substituted mono-, di- or tri-alkylamines, in particular mono-, di- or tri-alkylamines, or with quaternary ammonium compounds, for example with N-methyl-N-ethylamine, diethylamine, triethylamine, mono-, bis- or tris-(2-hydroxy-lower alkyl)amines, such as mono-, bis- or tris-(2-hydroxyethyl)amine, 2-hydroxy-tert-butylamine or tris(hydroxymethyl)methylamine, N,N-di-lower alkyl-N-(hydroxy-lower alkyl)amines, such as N,N-dimethyl-N-(2-hydroxyethyl)amine or tri-(2 hydroxyethyl)amine, or N-methyl-D-glucamine, or quaternary ammonium compounds such as tetrabutylammonium salts.

Exemplary compounds disclosed herein possess at least one basic group that can form acid-base salts with inorganic acids. Examples of basic groups include, but are not limited to, an amino group or imino group. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2- phenoxybenzoic acid, 2-acetoxybenzoic acid, embonic acid, nicotinic acid or isonicotinic acid, and, in addition, with amino acids, for example with a-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid.

Additional counterions for forming pharmaceutically acceptable salts are found in Remington's Pharmaceutical Sciences, 19th Edition, Mack Publishing Company, Easton, Pa., 1995. In one aspect, employing a pharmaceutically acceptable salt may also serve to adjust the osmotic pressure of a composition.

Tissue: A plurality of functionally related cells. A tissue can be a suspension, a semi-solid, or solid. Tissue includes cells collected from a subject such as the brain or a portion thereof.

Tumor: All neoplastic cell growth and proliferation, whether malignant or benign, and all pre-cancerous and cancerous cells and tissues. A primary tumor is tumor growing at the anatomical site where tumor progression began and proceeded to yield this mass. A primary brain tumor (also referred to as a glioma) is a tumor that originates in the brain. Exemplary primary brain tumors include astrocytomas, glioblastomas, ependymoma, oligodendroglomas, and mixed gliomas. In some examples, a primary brain tumor expresses susceptible receptor, such as a glioblastoma associated with susceptible receptor expression.

Under conditions sufficient for: A phrase that is used to describe any environment that permits the desired activity. In one example, under conditions sufficient for includes administering one or more cancer killer, cancer killer or a combination thereof to a subject to at a concentration sufficient to allow the desired activity. In some examples, the desired activity is reducing or inhibiting a sign or symptom associated with a disorder or disease, such as a primary brain tumor, hepatocellular carcinoma, liver cancer, colon cancer, or lung cancer, can be evidenced, for example, by a delayed onset of clinical symptoms of the tumor in a susceptible subject, a reduction in severity of some or all clinical symptoms of the tumor, a slower progression of the tumor (for example by prolonging the life of a subject having the tumor), a reduction in the number of tumor reoccurrence, an improvement in the overall health or well-being of the subject, or by other parameters well known in the art that are specific to the particular disease. In one particulate example, the desired activity is preventing or inhibiting tumor growth, such as astrocytoma, glioblastoma, or hepatocellular carcinoma growth. Tumor growth does not need to be completely inhibited for the treatment to be considered effective. For example, a partial reduction or slowing of growth such as at least about a 10% reduction, such as at least 20%, at least about 30%, at least about 40%, at least about 50% or greater is considered to be effective.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹³C). Isotopic substitution with ²H, ¹¹C, ¹³C, ¹³C, ¹⁴C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ²³S, ²⁴S, ³⁶S, ³⁵Cl, ³⁷Cl, ⁷⁹br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

### Chemical Structure

In some embodiments, the method includes administering to a subject having or at risk of developing a disorder or disease an effective amount of a compound to reduce one or more symptoms associated with the disorder or disease, wherein the compound has the general formula:

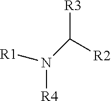

In some embodiments, R₁ and R₃ are either hydrogen or short alkyl groups. R₄ can be hydrogen, alkyl, aryl, or alkyl chains terminating in aryl substituents. In some embodiments, R₂ is strictly a naphthyl substituent (either 1- or 2- substituted), or may be separated by a short alkyl chain The naphthyl substituent is as shown:

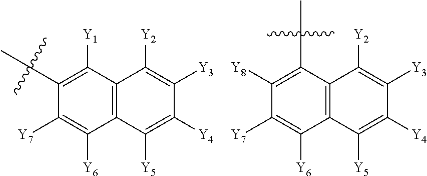

In some embodiments, Y₁-Y₈ independently are hydrogen, deuterium, halogen (F, Cl, Br, I), hydroxyl (—OH) or alkoxy (—OR), nitrogen substituents such as primary amines, substituted secondary and tertiary amines, sulfur-containing moieties including SH, sulfoxides, sulfones, sulfanamides and related alkyl and aryls.

In some embodiments, administering comprises administering a therapeutically effective amount of naphthyl isopropyl amine also known as PAL-287 described as NT015, NT016 or related compounds described in this document or a combination thereof.

In some embodiments, administering comprises administering a therapeutically effective amount of NT015, NT016 or related compounds described in this document or a combination thereof.

In some embodiments, the disorder or disease is liver cancer, brain cancer, lung cancer, breast cancer or any cancer that responds to these compounds.

In some embodiments, administration results in inhibiting one or more signs or symptoms associated with the disease or disorder comprises inhibiting cellular growth, such as tumor or cancer cell growth (or both), tumor volume, or a combination thereof.

In some embodiments, administering comprises of administering an additional therapeutic agent, such as prior to, concurrent with, or subsequent to administering a compound

In some embodiments, the additional therapeutic agent is a chemotherapeutic agent or agent with antitumor activity.

In some embodiments, administering a therapeutically effective amount of the compound includes the administration of a pharmaceutically acceptable carrier.

In some embodiments, the subject is human.

In some embodiments, The compounds described herein may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., March, Advanced Organic Chemistry, 4th edition, New York: John Wiley and Sons, 1992, Chapter 4).

In some embodiments, the method includes administering a therapeutically effective amount of a pharmaceutical composition containing any of the disclosed compounds capable of regulating a susceptible disorder or disease and a pharmaceutically acceptable carrier to treat the disorder or disease regulated any of the compounds, such as a glioblastoma or hepatocellular carcinoma expressing disease. For example, the disclosed compounds, or a combination thereof are effective at treating a glioblastoma or hepatocellular carcinoma expressing a disorder, such as a disease expressing glioblastoma or hepatocellular carcinoma. In some embodiments, the method further includes selecting a subject having or at risk of developing a disorder or disease regulated by the compounds. For example, a subject is selected for treatment by determining that the disorder or tumor is associated with the compounds, such as susceptible receptor expression. In one particular example, the method further includes selecting a subject with a disorder and/or disease, which is not associated with altered disorder function. For example, the disorder or disease does not respond to a treatment targeting disorder activity. In further examples, the method includes administering one or more therapeutic agents in addition to the compounds or combination thereof. The methods can include administration of the one or more therapeutic agents separately, sequentially or concurrently, for example in a combined composition compounds or combinations thereof.

In some embodiments, the method is for use in treating a tumor expressing a susceptible receptor. For example, the disorder or disease is selected from the group consisting of a primary brain tumor expressing a susceptible receptor, a glioblastoma expressing a susceptible receptor, a hepatocellular carcinoma expressing a susceptible receptor, colon cancer, liver cancer, and lung cancer.

In some embodiments, inhibiting one or more signs or symptoms associated with the disease or disorder comprises inhibiting cellular growth, such as tumor and/or cancer cell growth, tumor volume or a combination thereof.

In some embodiments, the method is used to treat a susceptible disease.

In one example R₃ is methyl and R₄ and R₁ are hydrogens. Y₄ or Y₅ may be hydrogen, methoxy, or methyl groups.

In some embodiments, R₁ and R₃ are either hydrogen or alkyl groups, R₄ is hydrogen or an alkyl chain terminating in a hydroxy substituted aryls, R₂ is a substituted or unsubstituted naphthyl group optionally linked by a substituted or unsubstituted alkyl chain and each Y₁-Y₈ are independently selected from hydrogen, hydroxyl, and alkoxy substituents

In one embodiment R₃, independently are hydrogen or a lower alkyl chain with or without hydroxyl group and further linked to phenol or benzyl ring; R₃ is a lower alkyl (such as, CH₃ or CH₂CH₃). R₄ contains a substituted aryl, wherein one or more functional groups is selected from the group consisting of —OR₆ and —NR₇R₈; wherein R₆ is independently hydrogen, lower alkyl, acyl, alkoxy carbonyl or amino carbonyl; R₇ and R₈ independently are hydrogen, lower alkyl, alkoxy carbonyl, acyl or amino carbonyl and wherein the compound is optically active.

In one embodiment, the compound is Naphthylisopropylamine (PAL-287), also denoted as NT015 and 1-(naphthalen-2-yl)propan-2-amineherein, as shown below.

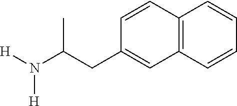

In another embodiment, the compound is 4-(2-(methyl(1-(naphthalen-1-yl)propan-2-yl)amino)ethyl)phenol), also denoted as NT016 as shown below.

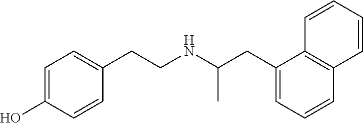 [SMILES: CC(Cc1cccc2ccccc12)NCCc1ccc(O)cc1]

In some embodiments, the compound is selected from the group consisting of NT009, NT010, NT011, NT017, NT018, NT019, NT020, NT021, NT022, NT023, NT026, NT027, NT029, NT030, NT034, NT035, NT037, NT038 and NT039, each shown in Table 1.

In some embodiments, the compound is selected from the group consisting of 1-(naphthalen-1-yl)propan-2-amine; 1-naphthalen-1-yl)ethan-1-amine; 1-(naphthalen-2-yl)ethan-1-amine; 1-(6-methoxynaphthalen-2-yl)ethan-1-amine; 1-(1-amino-2-methylpropyl)naphthalen-2-ol; 1-(naphthalen-2-yl)propan-2-amine; 4-(2-((1-(naphthalen-1-yl)propan-2-yl)amino)ethyl)phenol; 4-((1-(naphthalen-1-yl)propan-2-yl)amino)phenol; 4-((1-(naphthalen-2-yl)propan-2-yl)amino)phenol; 4-(1-hydroxy-2-((1-(naphthalen-1-yl)propan-2-yl)amino)ethyl)phenol; 2-((1-(naphthalen-2-yl)propan-2-yl)amino)-1-phenylethan-1-ol; 4-(2-((1-(naphthalen-2-yl)propan-2-yl)amino)ethyl)phenol; 3-(1-hydroxy-2-((1-(naphthalen-1-yl)propan-2-yl)amino)ethyl)phenol; 3-(1-hydroxy-2-((1-(naphthalen-2-yl)propan-2-yl)amino)ethyl)phenol; 4-(1-hydroxy-2-((1-(naphthalen-2-yl)propan-2-yl)amino)ethyl)benzene-1,2-diol; 4-(1-hydroxy-2-((1-(naphthalen-1-yl)propan-2-yl)amino)ethyl)benzene-1,2-diol; 4-(1-hydroxy-2-((1-(naphthalen-2-yl)propan-2-yl)amino)ethyl)phenol; 4-(1-hydroxy-2-((1-(naphthalen-1-yl)propan-2-yl)amino)ethyl)phenol; 2-(1-hydroxy-2-((1-(naphthalen-1-yl)propan-2-yl)amino)ethyl)phenol; 2-(1-hydroxy-2-((1-(naphthalen-2-yl)propan-2-yl)amino)ethyl)phenol; 4-(2-((1-(4-methylnaphthalen-1-yl)propan-2-yl)amino)ethyl)phenol; 4-(2-((1-(5-methylnaphthalen-1-yl)propan-2-yl)amino)ethyl)phenol; 4-(1-hydroxy-2-((1-(4-methylnaphthalen-1-yl)propan-2-yl)amino)ethyl)benzene-1,2-diol; 3-(1-hydroxy-2-((1-(4-methylnaphthalen-1-yl)propan-2-yl)amino)ethyl)phenol; 2-((1-(4-methylnaphthalen-1-yl)propan-2-yl)amino)-1-phenylethan-1-ol; 4-(1-hydroxy-2-((1-(4-methylnaphthalen-1-yl)propan-2-yl)amino)ethyl)phenol; 4-(((1-(4-methylnaphthalen-1-yl)propan-2-yl)amino)methyl)phenol; 4-(((1-(naphthalen-1-yl)propan-2-yl)amino)methyl)phenol and 4-(((1-(naphthalen-2-yl)propan-2-yl)amino)methyl)phenol.

Examples of suitable groups for R₁-R₃ that can be cleaved in vivo to provide a hydroxy group include, without limitation, acyl, acyloxy and alkoxy carbonyl groups. Compounds having such cleavable groups are referred to as “prodrugs.” The term “prodrug,” as used herein, means a compound that includes a substituent that is convertible in vivo (e.g., by hydrolysis) to a hydroxyl group. Various forms of prodrugs are known in the art, for example, as discussed in Bundgaard, (ed.), Design of Prodrugs, Elsevier (1985); Widder, et al. (ed.), Methods in Enzymology, Vol. 4, Academic Press (1985); Krogsgaard-Larsen, et al., (ed), Design and Application of Prodrugs, Textbook of Drug Design and Development, Chapter 5, 113 191 (1991); Bundgaard, et al., Journal of Drug Delivery Reviews, 8:1 38(1992); Bundgaard, Pharmaceutical Sciences, 77:285 et seq. (1988); and Higuchi and Stella (eds.) Prodrugs as Novel Drug Delivery Systems, American Chemical Society (1975).

In some embodiments, administering comprises administering a therapeutically effective amount of compound NT015, NT016 or other examples described herein or a combination thereof.

In some embodiments, the method includes administering a therapeutically effective amount of a pharmaceutical composition containing any of the disclosed compounds capable of regulating a susceptible disorder or disease and a pharmaceutically acceptable carrier to treat the disorder or disease regulated by a susceptible receptor, such as a glioblastoma or hepatocellular carcinoma expressing GPCR receptors including and not only GPR55, beta adrenergic receptors, or serotonergic receptors.

In certain embodiments, the compounds used in the method are provided are polymorphous. As such, the compounds can be provided in two or more physical forms, such as different crystal forms, crystalline, liquid crystalline or non-crystalline (amorphous) forms.

In one embodiment, the use of any of the compounds described herein such as NT015 or a hydrate or pharmaceutically acceptable salt thereof) or combinations thereof are intended for use in the manufacture of a medicament for regulation of a susceptible, GPCR receptor, in a subject either at risk of developing or having a susceptible receptor-regulated disorder (such as a metabolic, inflammatory, pain or the like disorder) or disease (such as hepatocellular carcinoma, glioblastoma, liver cancer, lung cancer, colon cancer, brain cancer, diabetes, or an inflammatory disease) modulated by GPCRs (such as GPR55, beta adrenergic receptors, or serotonergic receptors).

In one embodiment, the use of formulations suitable for such medicaments, subjects who may benefit from same and other related features are described elsewhere herein.

### Methods of Synthesis

The disclosed compounds can be synthesized by any method known in the art. Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein may be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via open column chromatography or prep chromatography.

### Synthesis of Compound 93 (NT016)

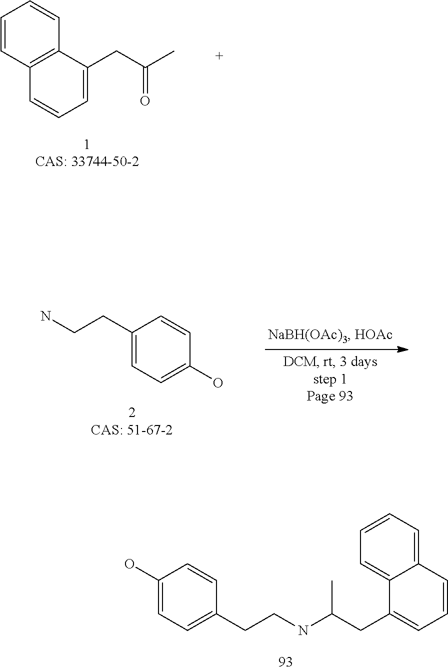 [SMILES: CC(=O)Cc1cccc2ccccc12 ; NCCc1ccc(O)cc1 ; CC(Cc1cccc2ccccc12)NCCc1ccc(O)cc1]

To the solution of 1 (184 mg, 1.0 mmol) and 2 (137 mg, 1.0 mmol) in DCM (10 mL), HOAc (120 mg, 2.0 mmol) was added, the mixture was stirred at rt for 30 min, then NaBH (OAc)₃ (424 mg, 2.0 mmol) was added portionwise. The final mixture was stirred over three days. Brine was added, then extracted by EtOAc (20 mL): washed by brine (30 mL×2), dried over Na₂SO₄. The crude product was purification by chromatography (silicone gel, CHCl₃:MeOH=20:1), to give 210 mg of white foam. HNMR (CDCl3, 300 MHz): 1.29 (dd, 3H, J1=15 Hz, J2=6 Hz), 2.90-3.31 (m, 5H), 3.53-3.57 (m, 1H), 3.84-3.90 (m, 1H), 6.72 (s, 4H), 7.32-7.34 (m, 2H), 7.47-7.53 (m, 2H), 7.52(d, 1H, J=3 Hz), 7.83(d, 1H, J=9 Hz), 8.14 (d, 1H, J=6 Hz).

### Synthesis of Compound 94 (NT017)

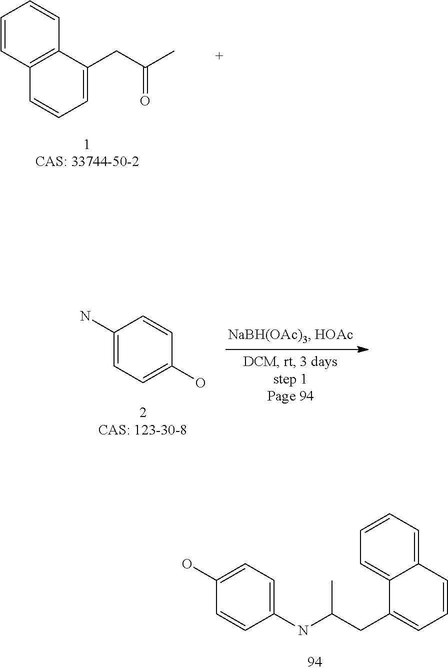 [SMILES: CC(=O)Cc1cccc2ccccc12 ; Nc1ccc(O)cc1 ; CC(Cc1cccc2ccccc12)Nc1ccc(O)cc1]

To the solution of 1 (184 mg, 1.0 mmol) and 2 (109 mg, 1.0 mmol) in DCM (10 mL), HOAc (120 mg, 2.0 mmol) was added, the mixture was stirred at rt for 30 min, then NaBH (OAc)₃ (424 mg, 2.0 mmol) was added portionwise. The final mixture was stirred over three days. Brine was added, then extracted by EtOAc (20 mL): washed by brine (30 mL×2), dried over Na₂SO₄. The crude product was purification by chromatography (silicone gel, CHCl₃:MeOH=20:1), to give 280 mg of yellow solid. HNMR (CDCl3, 300 MHz): 1.10 (d, 3H, J=6 Hz), 2.97-3.04 (m, 1H), 3.37-3.43 (m, 1H), 3.75-3.82 (m, 1H), 6.51-6.66 (m, 4H), 7.19-75 (m, 4H), 7.68 (d, 1H, J=9 Hz), 7.78 (d, 1H, J=9 Hz), 7.98 (d, 1H, J=9 Hz).

### Synthesis of Compound 102 (NT018)

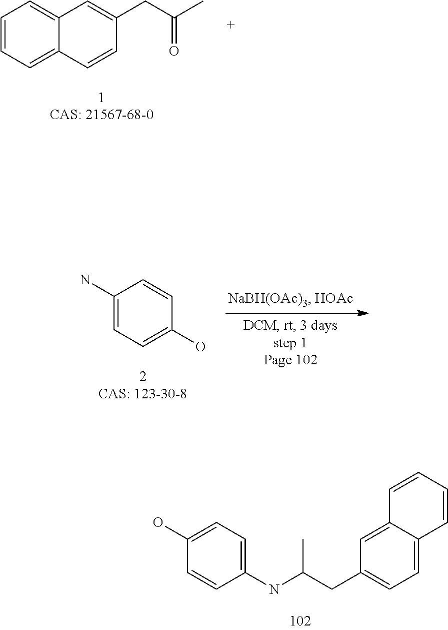 [SMILES: CC(=O)Cc1ccc2ccccc2c1 ; Nc1ccc(O)cc1 ; CC(Cc1ccc2ccccc2c1)Nc1ccc(O)cc1]

To the solution of 1 (184 mg, 1.0 mmol) and 2 (109 mg, 1.0 mmol) in DCM (2 mL), HOAc (120 mg, 2.0 mmol) was added, the mixture was stirred at rt for 30 min, then NaBH (OAc)₃ (424 mg, 2.0 mmol) was added portionwise. The final mixture was stirred over three days. Brine was added, then extracted by EtOAc(20 mL): washed by brine (30 mL×2), dried over Na₂SO₄. The crude product was purification by chromatography (silicone gel, ErOAc: Hexane=1:2 to 1:1), to give 231 mg of yellow solid. HNMR (CDCl3, 300 MHz): 1.19 (d, 3H, J=6 Hz), 2.83-2.90 (m, 1H), 3.09-3.15 (m, 1H), 3.77-3.83 (m, 1H), 6.61-6.77 (m, 4H), 7.33-36(m, 1H), 7.45-7.51 (m, 2H), 7.63 (s, 1H), 7.79-7.85 (m, 3H).

### Synthesis of Compound 95 (NT019)

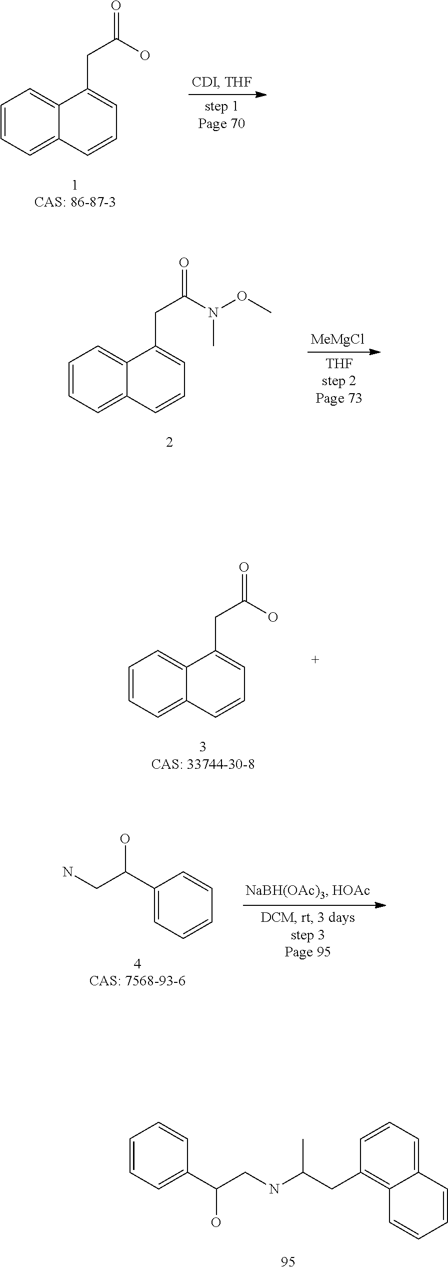 [SMILES: "MeMgCl" ; O=C(O)Cc1cccc2ccccc12 ; CON(C)C(=O)Cc1cccc2ccccc12 ; O=C(O)Cc1cccc2ccccc12 ; NCC(O)c1ccccc1 ; CC(Cc1cccc2ccccc12)NCC(O)c1ccccc1]

Step 1) synthesis of compound 2: To the suspension of N,O-Dimethylhydroxyamine hydrochloride (2.88 g, 29.6 mmol), then reagent 1 (5.0 g, 26.88 mmol) and triethylamine (2.98 g, 29.6 mmol) was added. After reaction mixture was stirred at room temperature for 20 min, CDI (4.79 g, 29.6 mmol) was added portionwise at room temperature. The final mixture was stirred further three hours. The mixture was poured into ice/water, extracted with EtOAc (100 ml×2), dried over Na₂SO₄. The organic solvent was evaporated under reduced pressure, the crude residue was used for the next step without further purification, 5.99 g as yellow oil.

Step 2) synthesis of compound 3: To the solution of 2 (6.0 g, 26.0 mmol) in dry THF (30 mL), MeMgCl (18.2 mL, 3M in THF) was added dropwise at 0° C., then the final mixture was stirred overnight at rt. The mixture was carefully quenched by saturated NH4Cl a.q., added EtOAc (200 mL), washed by brine (30 mL×2), 1N HCl (20 mL), brine (30 mL), dried over Na₂SO₄. The crude product was purification by chromatography (silicone gel, EtOAc:hexane=1:10), to give 4.2 g of 3 as yellow oil, yield 84.5% over two steps. HNMR (CDCl3, 300 MHz): 2.19 (s, 3H), 4.18 (s, 2H), 7.48-7.59 (m, 4H), 7.82-7.98 (m, 3H).

Step 3) synthesis of compound 95 (NT019): To the solution of 3 (184 mg, 1.0 mmol) and 4 (150 mg, 1.1 mmol) in DMF (2 mL), HOAc(120 mg, 2.0 mmol) was added, the mixture was stirred at rt for 30 min, then NaBH(OAc)₃ (424 mg, 2.0 mmol) was added portionwise. The final mixture was stirred over weekend. Brine was added, then extracted by EtOAc (20 mL): washed by brine (30 mL×2), dried over Na₂SO₄. The crude product was purification by chromatography (silicone gel, CHCl₃:MeOH=20:1), to give 360 mg of yellow oil. HNMR (CDCl3, 300 MHz): 1.25-1.32 (m, 3H), 3.24-3.37 (m, 3H), 3.67-3.69 (m, 1H), 3.97-4.09 (m, 1H), 5.44-5.52 (m, 1H), 7.20-7.35 (m, 10H), 7.42-7.74 (m, 2H), 7.81-8.01 (m, 2H), 8.15-8.29 (m, 1H).

### Synthesis of Compound 117 (NT020)

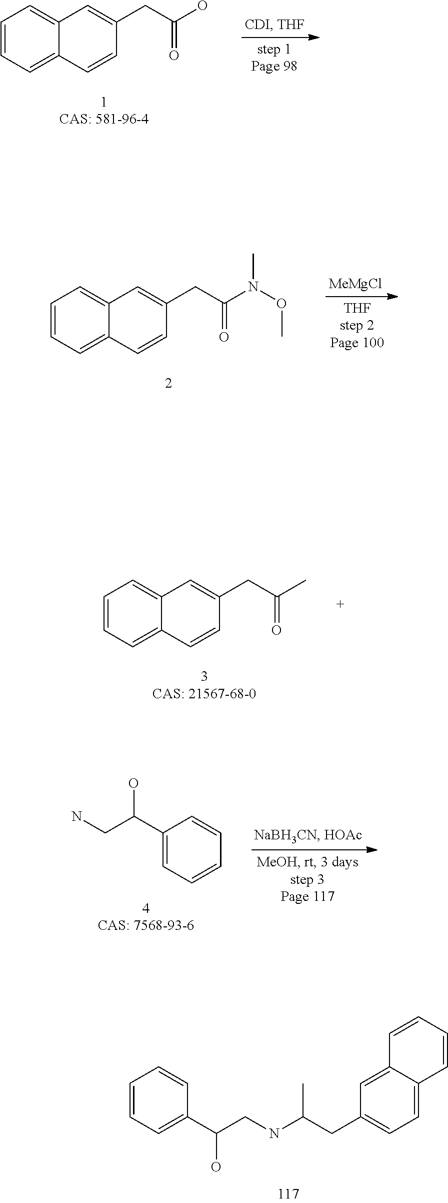 [SMILES: "MeMgCl" ; O=C(O)Cc1ccc2ccccc2c1 ; CON(C)C(=O)Cc1ccc2ccccc2c1 ; CC(=O)Cc1ccc2ccccc2c1 ; NCC(O)c1ccccc1 ; CC(Cc1ccc2ccccc2c1)NCC(O)c1ccccc1]

### Synthesis of Compound 117 (NT020)

Step 1) synthesis of compound 2: To the suspension of N,O-Dimethylhydroxyamine hydrochloride (2.79 g, 28.22 mmol) in THF (40 mL), then reagent 1 (5.0 g, 26.88 mmol) and triethylamine (2.98 g, 29.6 mmol) was added. After reaction mixture was stirred at room temperature for 20 min, CDI (4.57 g, 28.22 mmol) was added portionwise at room temperature. The final mixture was stirred further overnight. The mixture was poured into ice/water, extracted with EtOAc (100 ml×2), dried over Na₂SO₄. Evaporated under reduced pressure, the crude residue was used for the next step without further purification.

Step 2) synthesis of compound 3: To the solution of 2 (6.0 g, 26.0 mmol) in dry THF (30 mL), MeMgCl (18.0 mL, 3M in THF) was added dropwise at 0° C., then the final mixture was stirred overnight at rt. The mixture was carefully quenched by saturated NH4Cl a.q., added EtOAc (200 mL), washed by brine (30 mL×2), 1N HCl (20 mL), brine (30 mL), dried over Na₂SO₄. The crude product was purification by chromatography (silicone gel, EtOAc:hexane=1:10), to give 4.2 g of 3 as yellow oil, yield 80% over two steps. HNMR (CDCl3, 300 MHz): 2.21 (s, 3H), 3.88 (s, 2H), 7.35 (d, 1H, J-9 Hz), 7.48-7.51 (m, 2H), 7.70 (s, 1H), 7.81-7.86 (m, 3H).

Step 3) synthesis of compound 117: To the solution of 3 (100 mg, 0.54 mmol) and 4 (82 mg, 0.59 mmol) in MeOH (3 mL), HOAc (32 mg, 0.54 mmol) was added, the mixture was stirred at rt for 30 min, then NaBH₃CN (68 mg, 1.08 mmol) was added portionwise. The final mixture was stirred over weekend. Brine was added, then extracted by EtOAc (20 mL): washed by brine (30 mL×2), dried over Na₂SO₄. The crude product was purification by chromatography (silicone gel, EtOAc), to give 170 mg of 117 as yellow oil. HNMR (CDCl3, 300 MHz): 1.35-1.30 (m, 3H), 2.97-3.43 (m, 5H), 5.15-5.18 (m, 1H), 6.03 (m, 2H), 7.26-7.35 (m, 6H), 7.45-7.47 (m, 2H), 7.67 (s, 1H), 7.76-7.78 (m, 2H).

### Synthesis of Compound 103 (NT021)

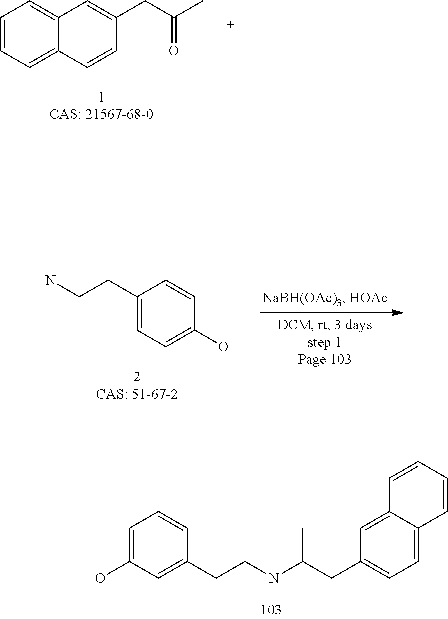 [SMILES: CC(=O)Cc1ccc2ccccc2c1 ; NCCc1ccc(O)cc1 ; CC(Cc1ccc2ccccc2c1)NCCc1cccc(O)c1]

To the solution of 1 (184 mg, 1.0 mmol) and 2 (137 mg, 1.0 mmol) in DCM (2 mL), HOAc (120 mg, 2.0 mmol) was added, the mixture was stirred at rt for 30 min, then NaBH (OAc)₃ (424 mg, 2.0 mmol) was added portionwise. The final mixture was stirred over three days. Brine was added, then extracted by EtOAc(20 mL): washed by brine (30 mL×2),dried over Na₂SO₄. The crude product was purification by chromatography (silicone gel, DCM:MeOH=100:1 to 50:1), to give 280 mg of yellow oil. HNMR (CDCl3, 300 MHz): 1.15(d, 3H, J=6 Hz), 2.64-3.04 (m, 7H), 3.53-3.59 (m, 1H), 3.84-3.90 (m, 1H), 6.57-6.60 (m, 2H), 6.78-6.88 (m, 3H), 7.04(d, 1H, J=9 Hz), 7.23-7.28 (m, 1H), 7.45-7.48 (m, 2H), 7.56 (s, 1H), 7.74-7.81 (m, 3H).

### Synthesis of Compound 96 (NT022)

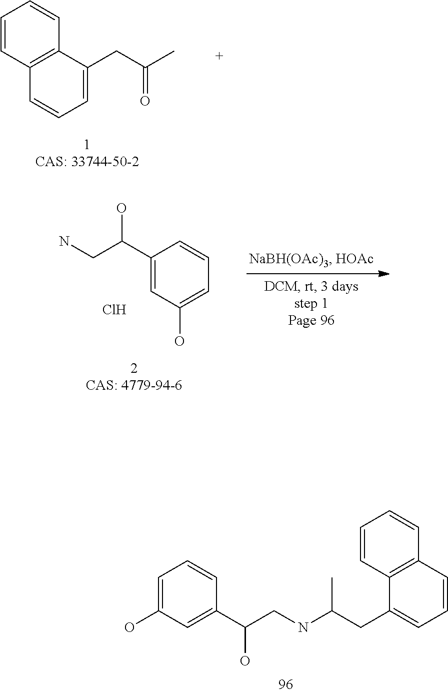 [SMILES: CC(=O)Cc1cccc2ccccc12 ; NCC(O)c1cccc(O)c1 ; Cl ; CC(Cc1cccc2ccccc12)NCC(O)c1cccc(O)c1]

To the solution of 1 (184 mg, 1.0 mmol) and 2 (208 mg, 1.1 mmol) in DCM (2 mL), HOAc (120 mg, 2.0 mmol) was added, the mixture was stirred at rt for 30 min, then NaBH (OAc)₃ (424 mg, 2.0 mmol) was added portionwise. The final mixture was stirred over three days. Brine was added, then extracted by EtOAc (20 mL): washed by brine (30 mL×2), dried over Na₂SO₄. The crude product was purification by chromatography (silicone gel, CHCl₃:MeOH=15:1 to 10:1), to give 350 mg of yellow oil. HNMR (CDCl3, 300 MHz): 1.01-1.06 (m, 3H), 2.65-3.30 (m, 4H), 4.63-4.73 (m, 1H), 5.44 (m, 2H), 6.70-6.79 (m, 2H), 6.89-6.91 (m, 1H), 7.06-7.47 (m, 5H), 7.30-7.97 (m, 3H).

### Synthesis of Compound 107 (NT023)

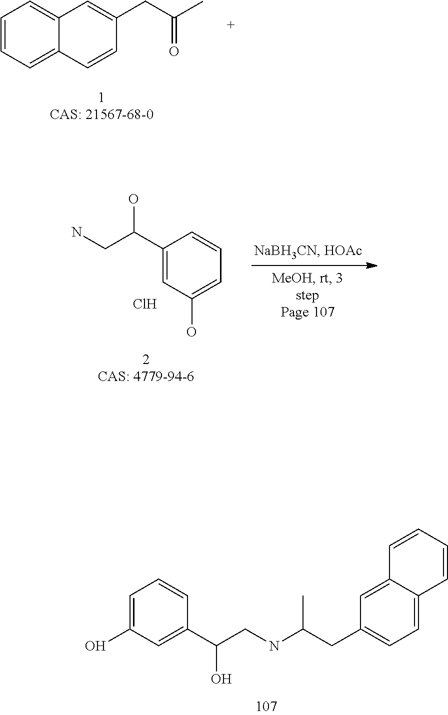 [SMILES: CC(=O)Cc1ccc2ccccc2c1 ; NCC(O)c1cccc(O)c1 ; Cl ; CC(Cc1ccc2ccccc2c1)NCC(O)c1cccc(O)c1]

To the solution of 1 (184 mg, 1.0 mmol) and 2 (189 mg, 1.1 mmol) in MeOH (3 mL), HOAc (120 mg, 2.0 mmol) was added, the mixture was stirred at rt for 30 min, then NaBH (OAc)₃ (424 mg, 2.0 mmol) was added portionwise. The final mixture was stirred over three days. Brine was added, then extracted by EtOAc (20 mL): washed by brine (30 mL×2), dried over Na₂SO₄. The crude product was purification by chromatography (silicone gel, EtOAc:Hexane=0:100 to 100:0), to give 160 mg of yellow oil. HNMR(CDCl3, 300 MHz): 1.13-1.06 (m, 3H), 2.79-3.33 (m, 3H), 3.43-3.61 (m, 2H), 4.83-4.86 (m, 1H), 6.22 (m, 1H), 6.71-6.87 (m, 3H), 7.19(t, 1H, J=6 Hz), 7.50-7.53 (m, 3H), 7.76-7.93 (m, 4H).

### Synthesis of Compound 119 (NT024)

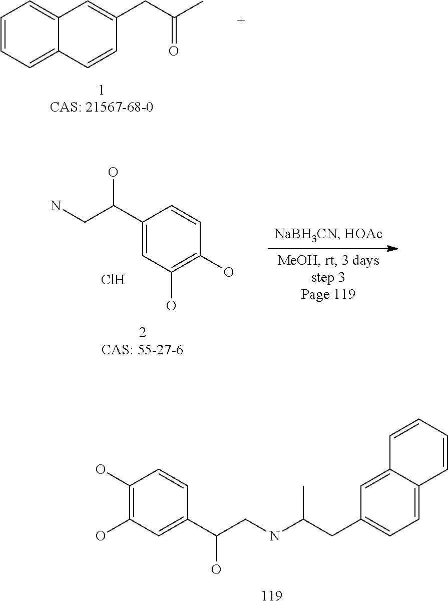 [SMILES: CC(=O)Cc1ccc2ccccc2c1 ; NCC(O)c1ccc(O)c(O)c1 ; Cl ; CC(Cc1ccc2ccccc2c1)NCC(O)c1ccc(O)c(O)c1]

To the solution of 1 (100 mg, 0.54 mmol) and 2 (121 mg, 0.59 mmol) in MeOH (3 mL), HOAc (120 mg, 2.0 mmol) and Et₃N (60 mg, 0.59 mmol) was added, the mixture was stirred at rt for 30 min, then NaBH₃CN (68 mg, 1.08 mmol) was added portionwise. The final mixture was stirred over three days. Brine was added, then extracted by EtOAc (20 mL): washed by brine (30 mL×2), dried over Na₂SO₄. The crude product was purification by chromatography (silicone gel, DCM:MeOH=100:1 to 30:1), to give 50 mg of white foam. HNMR (CDCl3, 300 MHz): 1.13 (d, 3H, J=6 Hz), 2.72-3.58 (m, 5H), 4.71-4.75 (m, 1H), 6.03 (s, 1H), 6.61-6.83 (m, 3H), 7.39-7.51 (m, 3H), 7.75-7.90 (m, 3H), 8.06 (s, 1H), 8.16 (d, 1H, J=6 Hz), 8.60 (s, 2H), 8.96 (s, 2H).

### Synthesis of Compound 122 (NT025)

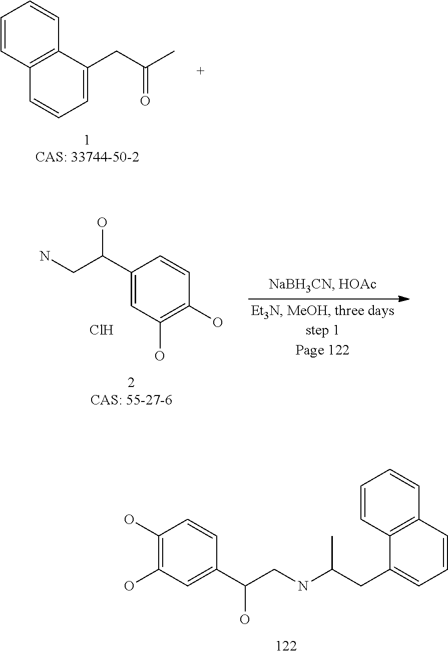 [SMILES: CC(=O)Cc1cccc2ccccc12 ; NCC(O)c1ccc(O)c(O)c1 ; Cl ; CC(Cc1cccc2ccccc12)NCC(O)c1ccc(O)c(O)c1]

To the solution of 1 (100 mg, 0.54 mmol) and 2 (121 mg, 0.59 mmol) in MeOH (2 mL), HOAc (120 mg, 2.0 mmol) and Et₃N (60 mg, 0.59 mmol) was added, the mixture was stirred at rt for 30 min, then NaBH₃CN (74 mg, 1.18 mmol) was added portionwise. The final mixture was stirred over three days. Brine was added, then extracted by EtOAc (20 mL): washed by brine (30 mL×2), dried over Na₂SO₄. The crude product was purification by chromatography (silicone gel, DCM:MeOH=100: lt to 50:1), to give 60 mg of white foam. HNMR (CDCl3, 300 MHz): 1.03 (d, 3H, J=6 Hz), 2.89-3.01 (m, 4H), 3.54 (m, 1H), 4.63 (m, 1H), 6.62-6.80 (m, 3H), 7.37-7.59 (m, 2H), 8.16 (d, 1H, J=6 Hz), 8.89 (s, 1H).

### Synthesis of Compound 118 (NT026)

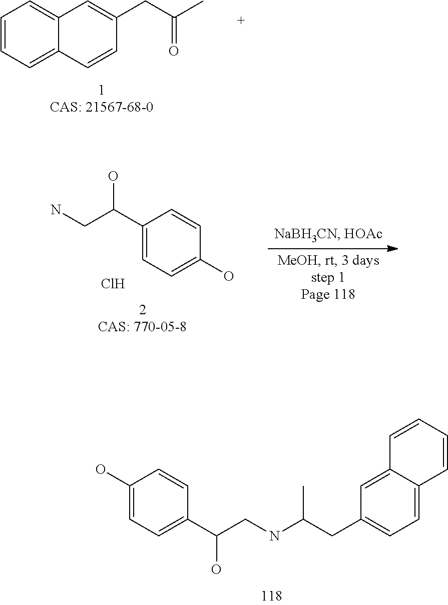 [SMILES: CC(=O)Cc1ccc2ccccc2c1 ; NCC(O)c1ccc(O)cc1 ; Cl ; CC(Cc1ccc2ccccc2c1)NCC(O)c1ccc(O)cc1]

To the solution of 1 (100 mg, 0.54 mmol) and 2 (112 mg, 0.59 mmol) in MeOH (3 mL), HOAc (120 mg, 2.0 mmol) and Et₃N (60 mg, 0.59 mmol) was added, the mixture was stirred at rt for 30 min, then NaBH₃CN (68 mg, 1.08 mmol) was added portionwise. The final mixture was stirred over three days. Brine was added, then extracted by EtOAc (20 mL): washed by brine (30 mL×2), dried over Na₂SO₄. The crude product was purification by chromatography (silicone gel, DCM:MeOH=20: It to 30:1), to give 160 mg of colorless oil. HNMR (DMSO, 300 MHz): 1.03 (d, 3H, J=6 Hz), 2.72-2.89 (m, 4H), 3.07-3.35 (m, 2H), 4.61-4.62 (m, 1H), 6.68-6.73 (m, 2H), 7.12-7.17 (m, 2H), 7.33-7.50 (m, 3H), 7.70 (s, 1H), 7.84-7.89 (m, 2H).

### Synthesis of Compound 123 (NT027)

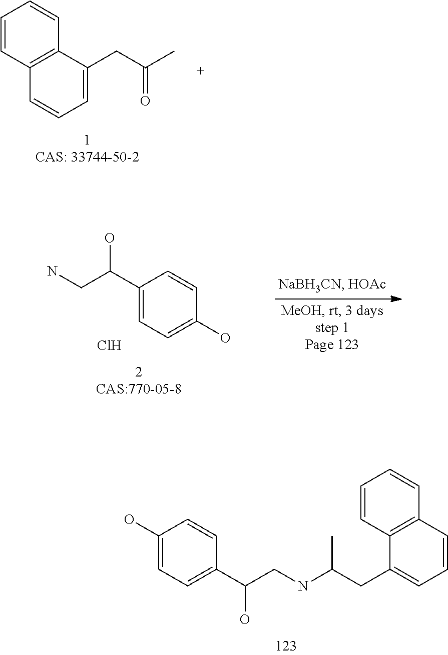 [SMILES: CC(=O)Cc1cccc2ccccc12 ; NCC(O)c1ccc(O)cc1 ; Cl ; CC(Cc1cccc2ccccc12)NCC(O)c1ccc(O)cc1]

To the solution of 1 (100 mg, 0.54 mmol) and 2 (112 mg, 0.59 mmol) in MeOH (2 mL), HOAc (120 mg, 2.0 mmol) and Et₃N (60 mg, 0.59 mmol) was added, the mixture was stirred at rt for 30 min, then NaBH₃CN (74 mg, 1.18 mmol) was added portionwise. The final mixture was stirred over three days. Brine was added, then extracted by EtOAc(20 mL): washed by brine (30 mL×2), dried over Na₂SO₄. The crude product was purification by chromatography (silicone gel, DCM:MeOH=100: lt to 30:1), to give 120 mg of yellow solid. HNMR (DMSO, 300 MHz): 0.99 (d, 3H, J=6 Hz), 2.68-3.01 (m, 5H), 4.52-4.57 (m, 1H), 6.67-6.72 (m, 2H), 7.08-7.15 (m, 2H), 7.34-7.56 (m, 4H), 7.55 (d, 1H, J=6 Hz), 7.80 (d, 1H, J=6 Hz), 8.10(d, 1H, J=6 Hz), 9.30 (s, 1H).

### Synthesis of Compound 136 (NT029)

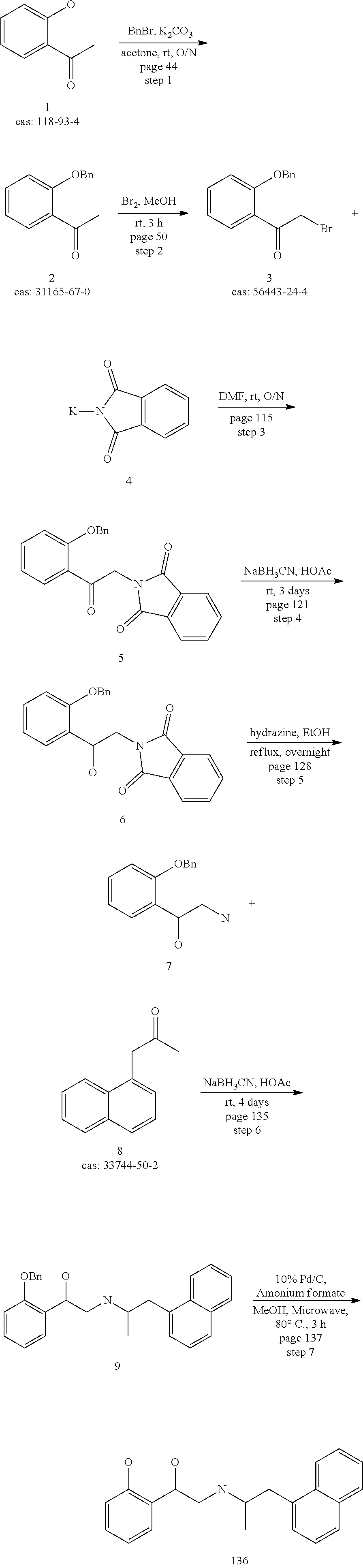

Step 1) synthesis of compound 2: To the solution of 1 (3.0 g, 22.06 mmol) and BnBr (3.8 g, 22.06 mmol) in acetone (30 mL), K2CO3 (3.04 g, 22.06 mmol) was added, the mixture was stirred at rt overnight. Excess of solvent was removed under reduced pressure, the residue was dissolved in EtOAc (100 mL), washed by brine, dried over Na₂SO₄. The crude product was purification by chromatography (silicone gel, EtOAc:hexane=1:15 to 1:10), to give 4.5 g of yellow oil.

Step 2) synthesis of compound 3: To the solution of 2 (1.0 g, 4.42 mmol) in MeOH (30 mL), bromine (0.74 g, 4.64 mmol) in MeOH (80 mL) was added dropwise over 1 hour period, the mixture was stirred at rt for 2 hours. Excess of solvent was removed under reduced pressure, the residue was dissolved in EtOAc (100 mL), washed by brine, dried over Na₂SO₄. Another batch with 3.3 g of 2 was synthesized under same set of conditions. The combined crude product (6.1 g) was used for the next step without further purification.

Step 3) synthesis of compound 5: To the solution of 3 (2.36 g, 7.74 mmol) in DMF (20 mL), compound 4 (1.57 g, 8.51 mmol) was added, the mixture was stirred at rt overnight. Excess of solvent was removed under reduced pressure, the residue was dissolved in EtOAc (150 mL), washed by brine, dried over Na₂SO₄. The crude product was purification by chromatography (silicone gel, EtOAc:hexane=1:10), to give 2.46 g of yellow solid.

Step 4) synthesis of compound 6: To the suspension of 5 (2.0 g, 5.39 mmol) in HOAc (10 mL), NaBH₃CN (0.68 g, 10.78 mmol) was added portionwise (totally for times), the mixture was stirred at rt for 3 days. Excess of solvent was removed under reduced pressure, the residue was dissolved in EtOAc (150 mL), washed by brine, saturated NaHCO3, dried over Na₂SO₄. The crude product was used for the next step without further purification.

Step 5) synthesis of compound 8: To the suspension of 6 (2.0 g, 5.39 mmol) in EtOH (100 mL), hydrazine (0.68 g, 21.56 mmol) was added, the mixture was stirred at 60° C. overnight. The mixture was cooled to ambient temperature, excess of solvent was removed under reduced pressure, the residue was dissolved in EtOAc (150 mL), washed by brine, dried over Na₂SO₄. The crude product was purification by chromatography (silicone gel, MeOH:CHCl₃=1:10), to give 0.75 g of yellow solid.

Step 6) synthesis of compound 9: To the suspension of 8 (150 mg, 0.81 mmol) and 7 (216 mg, 0.89 mmol) in DCM (3 mL), HOAc (97 mg, 1.62 mmol) and NaBH (OAc)₃ (343 mg, 1.62 mmol) was added, the final mixture was stirred at rt for 3 days. The crude product was purification by chromatography (silicone gel, MeOH:CHCl₃=1:50), to give 60 mg of yellow oil.

Step 7) synthesis of compound 136: To the suspension of 9 (60 mg) MeOH (2 mL), 10% Pd/C (20 mg) and ammonium formate (100 mg) was added. The mixture was heated under microwave condition for 3 h at 80° C. The excess of solvent was removed under reduced pressure, the residue was purified by chromatography (DCM:MeOH=50:1 to 20:1), to give 27 mg of colorless oil. HNMR (CDCl3, 300 MHz): 1.12-1.15 (m, 3H), 2.67-3.21 (m, 6H), 4.73-4.82 (m, 1H), 6.78-6.80 (m, 1H), 6.91-6.94 (m, 3H), 7.03-7.05 (m, 1H), 7.36-7.52 (m, 3H), 7.76-7.78 (m, 1H), 7.85-7.87 (m, 1H), 7.99-8.02 (m, 1H).

### Synthesis of Compound 137 (NT030)

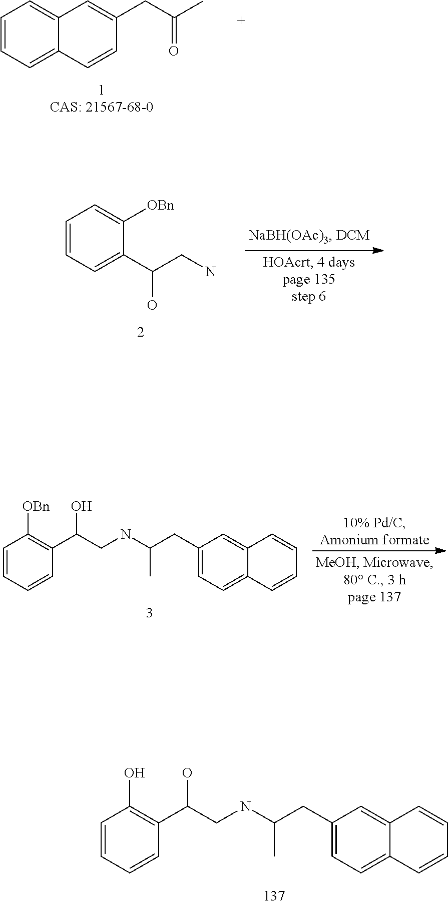 [SMILES: CC(=O)Cc1ccc2ccccc2c1 ; NCC(O)c1ccccc1OCc1ccccc1 ; CC(Cc1ccc2ccccc2c1)NCC(O)c1ccccc1OCc1ccccc1 ; CC(Cc1ccc2ccccc2c1)NCC(O)c1ccccc1O]

Step 1) synthesis of compound 3: To the suspension of 1 (150 mg, 0.81 mmol) and 2 (216 mg, 0.89 mmol) in DCM (3 mL), HOAc (97 mg, 1.62 mmol) and NaBH (OAc)₃ (343 mg, 1.62 mmol) was added, the final mixture was stirred at rt for 3 days. The crude product was purification by chromatography (silicone gel, MeOH:CHCl₃=1:50), to give 68 mg of 3 as yellow oil.

Step 2) synthesis of compound 137: To the suspension of 3 (68 mg) MeOH (2 mL), 10% Pd/C (20 mg) and ammonium formate (100 mg) was added. The mixture was heated under microwave condition for 3 h at 80° C. The crude product was purification by chromatography (silicone gel, MeOH:CHCl₃=1:50), to give 18 mg of yellow oil. HNMR (CDCl3, 300 MHz): 1.06 (d, 3H, J=6 Hz), 2.66-3.19 (m, 6H), 4.92-5.00 (m, 1H), 6.67-6.70 (m, 1H), 6.81-6.83 (m, 2H), 6.91-7.02 (m, 5H), 7.15-7.17 (m, 1H), 7.33-7.36 (m, 2H), 7.45-7.47 (m, 1H), 7.63-7.69 (m, 3H).

### Synthesis of Compound 175 (NT034)

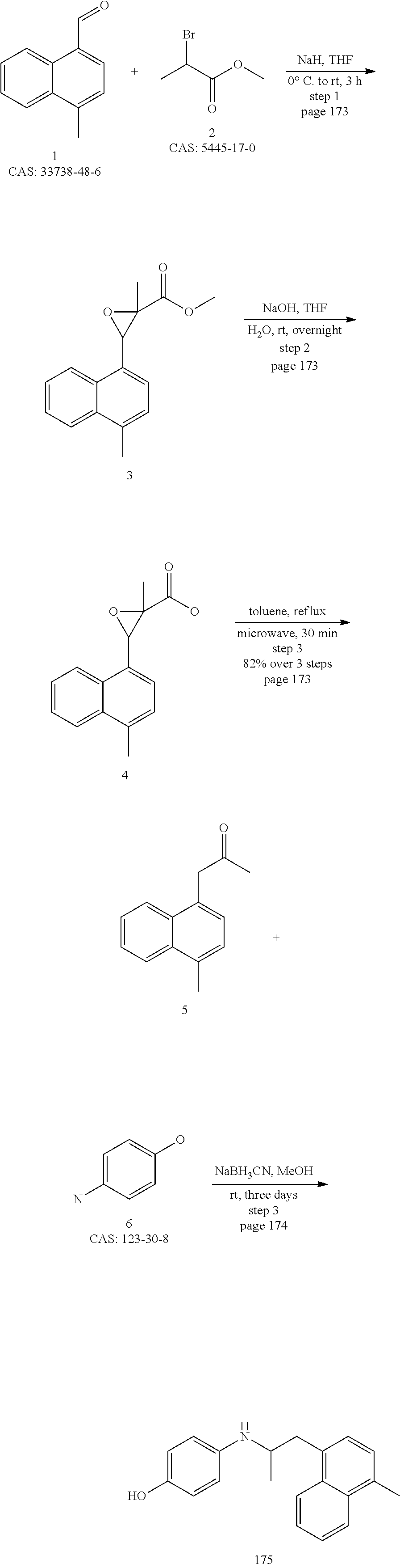

Step 1) synthesis of compound 3: To the suspension of sodium hydride (338 mg, 14.1 mmol) in THF (20 mL), then the solution of 2 (1.18 g, 7.05 mmol) in THF (5 mL) was added dropwise at 0° C. After reaction mixture was stirred for 10 min, the solution of 1 (800 mg, 4.76 mmol) in THF (5 mL) was added dropwise at 0° C. The final mixture was stirred further three hours. The crude mixture was used for the next step without further purification.

Step 2) synthesis of compound 4: After cooled to 0° C. again, the NaOH solution (20 mL, 2N) was added carefully, the resulting mixture was stirred at room temperature overnight. Excess of organic solvent was removed under reduced pressure, the residue was added EtOAc (100 mL), adjusted pH=3-5 by 1N HCl. Then the organic phase was washed by brine, dried over Na₂SO₄.he crude mixture was used for the next step without further purification.

Step 3) synthesis of compound 5: The above crude mixture was suspended in the mixture of toluene (2 mL) and water (0.5 mL), and then heated to 150° C. under microwave irradiation for 30 min. The crude product was purified by chromatography (EtOAc:hexane=1:30 to 1:20), to give 960 mg of colorless oil, 82% yield over three steps. HNMR (CDCl3, 300 MHz): 2.17 (s, 3H), 2.64 (s, 3H), 7.29 (m, 2H), 7.53-7.54 (m, 2H), 7.84-7.87 (m, 1H), 8.02-8.04 (m, 1H).

Step 4) synthesis of compound 175: To the solution of 5 (80 mg, 0.40 mmol) and 6 (48 mg, 0.44 mmol) in MeOH (2 mL), HOAc (48 mg, 0.8 mmol) was added, the mixture was stirred at rt for 30 min, then NaBH₃CN (50.4 mg, 0.80 mmol) was added. The final mixture was stirred over weekend. Brine was added, then extracted by EtOAc (20 mL): washed by brine (30 mL×2), dried over Na₂SO₄. The crude product was purification by chromatography (silicone gel, EtOAc:hexane=1:3), to give 110 mg of white solid. HNMR (DMSO, 300 MHz): 1.03 (d, 3H, J=6 Hz), 2.61 (s, 3H), 2.94-3.01 (m, 1H), 3.25-3.34 (m, 1H), 3.62-3.64 (m, 1H), 4.78-4.79 (m, 1H), 6.44-6.56 (m, 4H), 7.29 (m, 2H), 7.54-7.57 (m, 2H), 8.00-8.10 (m, 2H), 8.42 (s, 1H).

### Synthesis of Compound 176 (NT035)

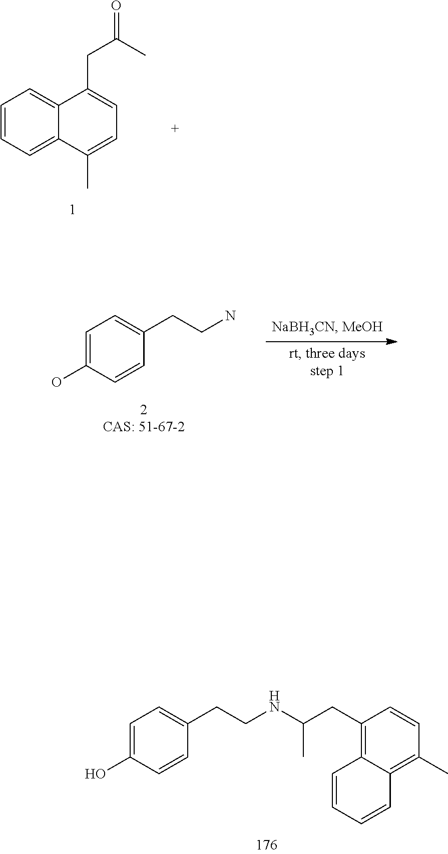 [SMILES: CC(=O)Cc1ccc(C)c2ccccc12 ; NCCc1ccc(O)cc1 ; Cc1ccc(CC(C)NCCc2ccc(O)cc2)c2ccccc12]

To the solution of 1 (80 mg, 0.40 mmol) and 2 (60 mg, 0.44 mmol) in MeOH (2 mL), HOAc (48 mg, 0.8 mmol) was added, the mixture was stirred at rt for 30 min, then NaBH₃CN (50 mg, 0.80 mmol) was added. The final mixture was stirred over three days. Brine was added, then extracted by EtOAc(20 mL): washed by brine (30 mL×2),dried over Na₂SO₄. The crude product was purification by chromatography (silicone gel, CHCl₃:MeOH=30:1, to give 108 mg of 176 as colorless oil. HNMR (DMSO, 300 MHz): 1.07 (d, 3H, J=6 Hz), 2.61 (s, 3H), 2.79-3.22(m, 5H), 3.48-3.64 (m, 2H), 6.72-6.75 (m, 2H), 7.06-7.09 (m, 2H), 7.26-7.35 (m, 2H), 7.53-7.63 (m, 2H), 9.34 (s, 1H).

### Synthesis of Compound 177 (NT036)

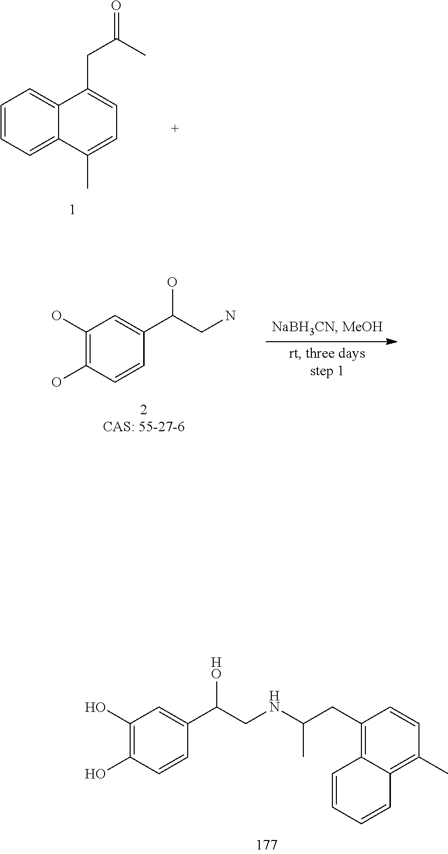 [SMILES: CC(=O)Cc1ccc(C)c2ccccc12 ; NCC(O)c1ccc(O)c(O)c1 ; Cc1ccc(CC(C)NCC(O)c2ccc(O)c(O)c2)c2ccccc12]

To the solution of 1 (80 mg, 0.40 mmol) and 2 (90 mg, 0.44 mmol) in MeOH (2 mL), HOAc (48 mg, 0.8 mmol) and Et₃N (45 mg, 0.44 mmol) was added, the mixture was stirred at rt for 30 min, then NaBH₃CN (50 mg, 0.80 mmol) was added. The final mixture was stirred over three days. Brine was added, then extracted by EtOAc (20 mL): washed by brine (30 mL×2), dried over Na₂SO₄. The crude product was purification by chromatography (silicone gel, CHCl₃:MeOH=30:1 to 15:1), to give 110 mg of colorless oil. HNMR (DMSO, 300 MHz): 1.05 (d, 3H, J=6 Hz), 2.63 (s, 3H), 2.95-3.12 (m, 3H), 3.46-3.61 (m, 2H), 4.67-4.73 (m, 1H), 6.62-6.82 (m, 3H), 7.29-7.31 (m, 2H), 7.58-7.61 (m, 2H), 8.04-8.20 (m, 2H), 8.90(s, 2H).

### Synthesis of Compound 178 (NT037)

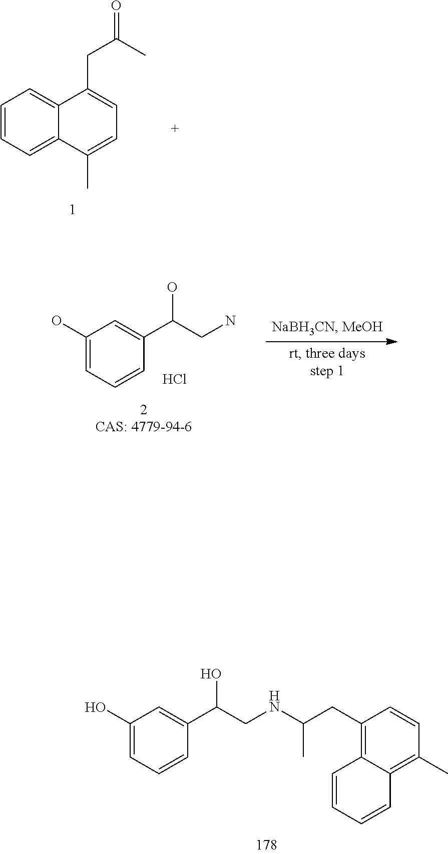 [SMILES: CC(=O)Cc1ccc(C)c2ccccc12 ; NCC(O)c1cccc(O)c1 ; Cl ; Cc1ccc(CC(C)NCC(O)c2cccc(O)c2)c2ccccc12]

To the solution of 1 (80 mg, 0.40 mmol) and 2 (84 mg, 0.44 mmol) in MeOH (2 mL), HOAc (48 mg, 0.8 mmol) and Et₃N (45 mg, 0.44 mmol) was added, the mixture was stirred at rt for 30 min, then NaBH₃CN (50 mg, 0.80 mmol) was added. The final mixture was stirred over three days. Brine was added, then extracted by EtOAc (20 mL): washed by brine (30 mL×2), dried over Na₂SO₄. The crude product was purification by chromatography (silicone gel, CHCl₃:MeOH=20:1), to give 80 mg of colorless oil. HNMR (DMSO, 300 MHz): 1.05 (d, 3H, J=6 Hz), 2.63 (s, 3H), 2.95-3.12 (m, 3H), 3.46-3.61 (m, 2H), 4.67-4.73 (m, 1H), 6.62-6.82 (m, 3H), 7.29-7.31 (m, 2H), 7.58-7.61 (m, 2H), 8.04-8.20 (m, 2H), 8.90 (s, 2H).

### Synthesis of Compound 179 (NT038)

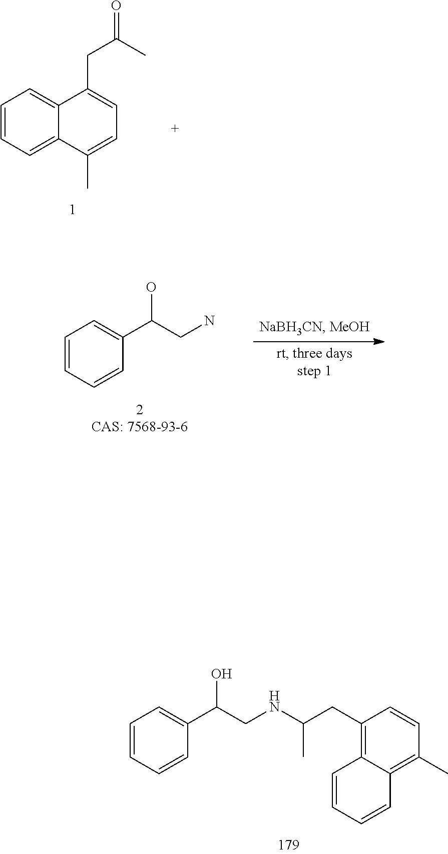 [SMILES: CC(=O)Cc1ccc(C)c2ccccc12 ; NCC(O)c1ccccc1 ; Cc1ccc(CC(C)NCC(O)c2ccccc2)c2ccccc12]

To the solution of 1 (80 mg, 0.40 mmol) and 2 (80 mg, 0.44 mmol) in MeOH (2 mL), HOAc (48 mg, 0.8 mmol) was added, the mixture was stirred at rt for 30 min, then NaBH₃CN (50 mg, 0.80 mmol) was added. The final mixture was stirred over three days. Brine was added, then extracted by EtOAc(20 mL): washed by brine (30 mL×2), dried over Na₂SO₄. The crude product was purification by chromatography (silicone gel, DCM:MeOH=10:1), to give 120 mg of 179 as colorless oil. HNMR (DMSO, 300 MHz): 1.06 (d, 3H, J=6 Hz), 2.63 (s, 3H), 2.93-3.07 (m, 3H), 3.14-3.60 (m, 2H), 4.80-4.89 (m, 1H), 7.27-7.41 (m, 9H), 8.03-8.06 (m, 1H), 8.18-8.19 (m, 1H).

### Synthesis of Compound 180 (NT039)

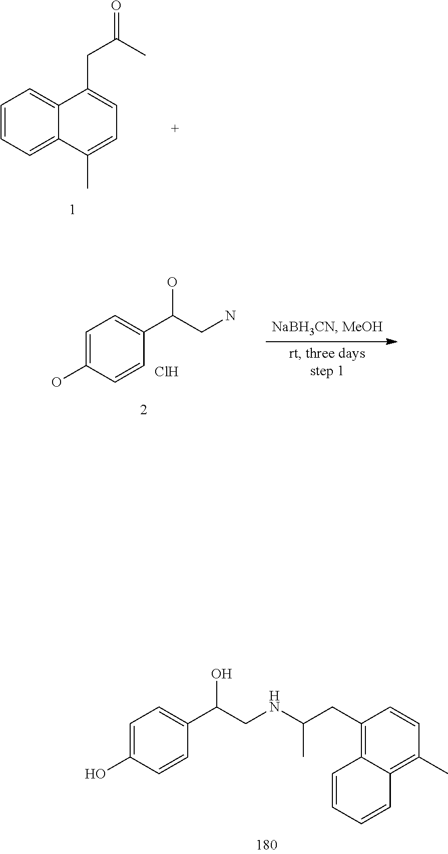 [SMILES: CC(=O)Cc1ccc(C)c2ccccc12 ; NCC(O)c1ccc(O)cc1 ; Cl ; Cc1ccc(CC(C)NCC(O)c2ccc(O)cc2)c2ccccc12]

To the solution of 1 (80 mg, 0.40 mmol) and 2 (84 mg, 0.44 mmol) in MeOH (2 mL), HOAc (48 mg, 0.8 mmol) and Et₃N (45 mg, 0.44 mmol) was added, the mixture was stirred at rt for 30 min, then NaBH₃CN (50 mg, 0.80 mmol) was added. The final mixture was stirred over three days. Brine was added, then extracted by EtOAc (20 mL): washed by brine (30 mL×2), dried over Na₂SO₄. The crude product was purification by chromatography (silicone gel, CHCl₃:MeOH=20:1), to give 80 mg of colorless oil. HNMR (DMSO, 300 MHz): 1.08 (d, 3H, J=6 Hz), 2.64 (s, 3H), 3.05-3.13 (m, 3H), 3.56-3.75 (m, 2H), 4.79-4.85 (m, 1H), 6.79-6.81 (m, 2H), 7.23-7.32 (m, 4H), 7.61 (m, 2H), 8.04-8.07 (m, 1H), 8.20-8.22 (m, 1H), 9.47 (s, 1H).

### Synthesis of Compound 201 (NT040)

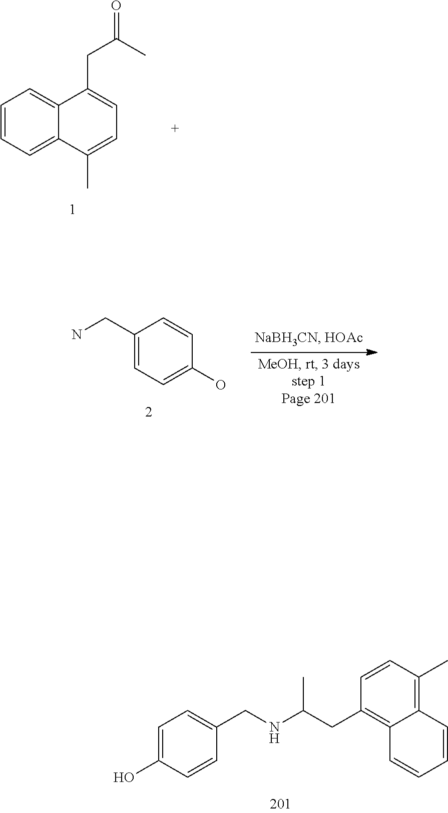 [SMILES: CC(=O)Cc1ccc(C)c2ccccc12 ; NCc1ccc(O)cc1 ; Cc1ccc(CC(C)NCc2ccc(O)cc2)c2ccccc12]

To the solution of 1 (100 mg, 0.50 mmol) and 2 (65 mg, 0.53 mmol) in MeOH (2 mL), HOAc (120 mg, 2.0 mmol) was added, the mixture was stirred at rt for 30 min, then NaBH₃CN (126 mg, 2.0 mmol) was added portionwise. The final mixture was stirred over weekend. Brine was added, then extracted by EtOAc (20 mL): washed by brine (30 mL×2), dried over Na₂SO₄. The crude product was purification by chromatography (silicone gel, CHCl₃:MeOH=40:1 to 20:1), to give 85 mg of colorless oil. HNMR(CDCl3, 300 MHz): 1.07 (d, 3H, J=6 Hz), 2.64 (s, 3H), 2.89-3.64 (m, 5H), 3.67-3.69 (m, 1H), 6.79-6.82 (m, 2H), 7.28-7.30 (m, 4H), 7.56 (m, 2H), 8.04 (m, 2H), 9.60 (m, 1H).

### Synthesis of Compound 202 (NT041)

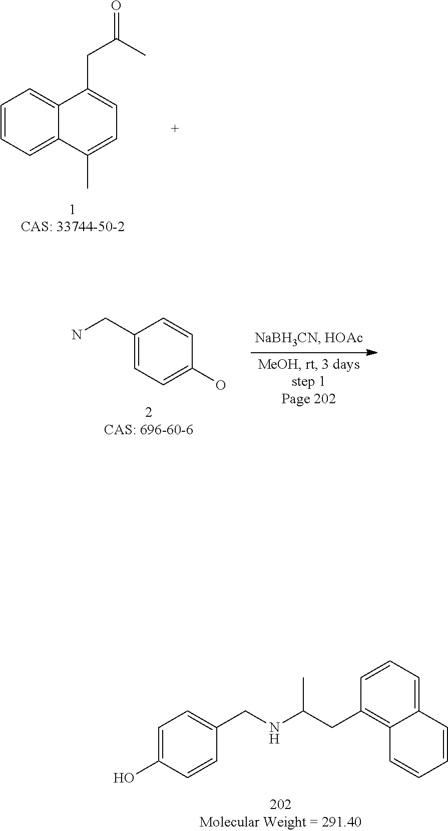 [SMILES: CC(=O)Cc1ccc(C)c2ccccc12 ; NCc1ccc(O)cc1 ; CC(Cc1cccc2ccccc12)NCc1ccc(O)cc1]

To the solution of 1 (100 mg, 0.54 mmol) and 2 (70 mg, 0.57 mmol) in MeOH (2 mL), HOAc (130 mg, 2.16 mmol) was added, the mixture was stirred at rt for 30 min, then NaBH₃CN (136 mg, 2.16 mmol) was added portionwise. The final mixture was stirred over three days. Brine was added, then extracted by EtOAc (20 mL): washed by brine (30 mL×2),dried over Na₂SO₄. The crude product was purification by chromatography (silicone gel, CHCl₃:MeOH=20:1), to give 80 mg of 202 as yellow oil. HNMR (DMSO-d6, 300 MHz): 0.98(d, 3H, J=6 Hz), 2.84-2.91 (m, 1H), 3.25-3.26 (m, 1H), 3.52-3.57 (m, 1H), 3.89-3.97 (m, 2H), 6.69-6.72 (m, 2H), 7.17-7.20 (m, 2H), 7.29-7.46 (m, 4H), 7.73-7.96 (m, 3H), 9.50 (m, 1H).

### Synthesis of Compound 203 (NT042)

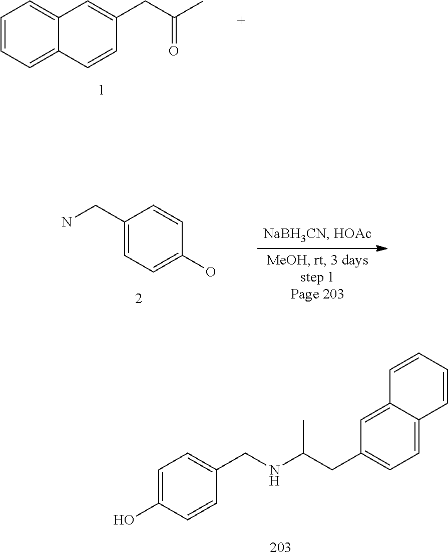 [SMILES: CC(=O)Cc1ccc2ccccc2c1 ; NCc1ccc(O)cc1 ; CC(Cc1ccc2ccccc2c1)NCc1ccc(O)cc1]

To the solution of 1 (100 mg, 0.54 mmol) and 2 (70 mg, 0.57 mmol) in MeOH (2 mL), HOAc (130 mg, 2.16 mmol) was added, the mixture was stirred at rt for 30 min, then NaBH₃CN (136 mg, 2.16 mmol) was added portionwise. The final mixture was stirred over weekend. Brine was added, then extracted by EtOAc (20 mL): washed by brine (30 mL×2), dried over Na₂SO₄. The crude product was purification by chromatography (silicone gel, CHCl₃:MeOH=40:1 to 20:1), to give 110 mg of colorless oil. HNMR (CDCl3, 300 MHz): 1.17 (d, 3H, H=6 Hz), 2.74-2.81 (m, 1H), 3.34-3.46 (m, 2H), 4.15 (s, 2H), 6.82-6.84 (m, 2H), 7.30-7.50 (m, 5H), 7.76-7.91 (m, 4H), 9.66 (m, 1H).

### Pharmaceutical Compositions

The disclosed compounds can be useful, at least, for reducing or inhibiting one or more symptoms or signs associated with a disorder (such as a metabolic, inflammatory, pain or the like disorder) or disease (such as hepatocellular carcinoma, glioblastoma, liver cancer, lung cancer, colon cancer, brain cancer, diabetes, or an inflammatory disease) modulated by cannabinoid receptors (such as GPR55). Accordingly, pharmaceutical compositions comprising at least one disclosed compounds are also described herein.

Formulations for pharmaceutical compositions are well known in the art. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of disclosed compounds. Pharmaceutical compositions comprising at least one of these compounds can be formulated for use in human or veterinary medicine. Particular formulations of a disclosed pharmaceutical composition may depend, for example, on the mode of administration (e.g., oral or parenteral) and/or on the disorder to be treated (e.g., a tumor associated with susceptible receptor, such as GPR55 receptor, activity or expression). In some embodiments, formulations include a pharmaceutically acceptable carrier in addition to at least one active ingredient, such as NT015, NT016 or a combination thereof.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions such as powder, pill, tablet, or capsule forms conventional non- toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances or excipients, such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations. [0151] The disclosed pharmaceutical compositions may be formulated as a pharmaceutically acceptable salt. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydriodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, ptoluenesulfonic acid, naphthalenesulfonic acid, and the like. Lists of other suitable pharmaceutically acceptable salts are found in Remington's Pharmaceutical Sciences, 19th Edition, Mack Publishing Company, Easton, Pa., 1995. A pharmaceutically acceptable salt may also serve to adjust the osmotic pressure of the composition.

The dosage form of a disclosed pharmaceutical composition will be determined by the mode of administration chosen. For example, in addition to injectable fluids, oral dosage forms may be employed. Oral formulations may be liquid such as syrups, solutions or suspensions or solid such as powders, pills, tablets, or capsules. Methods of preparing such dosage forms are known, or will be apparent, to those skilled in the art.

Certain embodiments of the pharmaceutical compositions comprising a disclosed compound may be formulated in unit dosage form suitable for individual administration of precise dosages. The amount of active ingredient such as NT015 or NT016 administered will depend on the subject being treated, the severity of the disorder, and the manner of administration, and is known to those skilled in the art. Within these bounds, the formulation to be administered will contain a quantity of the extracts or compounds disclosed herein in an amount effective to achieve the desired effect in the subject being treated.

In particular examples, for oral administration the compositions are provided in the form of a tablet containing from about 1.0 to about 50 mg of the active ingredient, particularly about 2.0 mg, about 2.5 mg, 5 mg, about 10 mg, or about 50 mg of the active ingredient for the symptomatic adjustment of the dosage to the subject being treated. In one exemplary oral dosage regimen, a tablet containing from about 1 mg to about 50 mg (such as about 2 mg to about 10 mg) active ingredient is administered two to four times a day, such as two times, three times or four times.

In other examples, a suitable dose for parental administration is about 1 milligram per kilogram (mg/kg) to about 100 mg/kg, such as a dose of about 10 mg/kg to about 80 mg/kg, such including about 1 mg/kg, about 2 mg/kg, about 5 mg/kg, about 20 mg/kg, about 30 mg/kg, about 40 mg/kg, about 50 mg/kg, about 80 mg/kg or about 100 mg/kg administered parenterally. However, other higher or lower dosages also could be used, such as from about 0.001 mg/kg to about 1 g/kg, such as about 0.1 to about 500 mg/kg, including about 0.5 mg/kg to about 200 mg/kg.

Single or multiple administrations of the composition comprising one or more of the disclosed compositions can be carried out with dose levels and pattern being selected by the treating physician. Generally, multiple doses are administered. In a particular example, the composition is administered parenterally once per day. However, the composition can be administered twice per day, three times per day, four times per day, six times per day, every other day, twice a week, weekly, or monthly. Treatment will typically continue for at least a month, more often for two or three months, sometimes for six months or a year, and may even continue indefinitely, i.e., chronically. Repeat courses of treatment are also possible.

In one embodiment, the pharmaceutical composition is administered without concurrent administration of a second agent for the treatment of a tumor that expresses a susceptible receptor, such as GPR55. In one specific, non-limiting example, one or more of the disclosed compositions is administered without concurrent administration of other agents, such as without concurrent administration of an additional agent also known to target the tumor. In other specific non-limiting examples, a therapeutically effective amount of a disclosed pharmaceutical composition is administered concurrently with an additional agent, including an additional therapy (such as, but not limited to, a chemotherapeutic agent, an additional regulator of susceptible receptor (such as regulator of GPR55), an anti-inflammatory agent, an anti-oxidant, or other agents known to those of skill in the art). For example, the disclosed compounds are administered in combination with a chemotherapeutic agent, anti-oxidants, anti-inflammatory drugs or combinations thereof.

In other examples, a disclosed pharmaceutical composition is administered an adjuvant therapy. For example, a pharmaceutical composition containing one or more of the disclosed compounds is administered orally daily to a subject in order to prevent or retard tumor growth. In one particular example, a composition containing equal portions of two or more disclosed compounds is provided to a subject. In one example, a composition containing unequal portions of two or more disclosed compounds is provided to the subject. For example, a composition contains unequal portions of compound . In one particular example, the composition includes a greater amount of the compound derivative. Such therapy can be given to a subject for an indefinite period of time to inhibit, prevent, or reduce tumor reoccurrence.

### Methods of Use

The present disclosure includes methods of treating disorders including reducing or inhibiting one or more signs or symptoms associated with a disorder (such as a metabolic, inflammatory, pain or the like disorder) or disease (such as hepatocellular carcinoma, glioblastoma, liver cancer, lung cancer, colon cancer, brain cancer, diabetes, or an inflammatory disease) modulated by GPCRs such as cannabinoid receptors (such as GPR55), beta adrenergic receptors, and serotonergic receptors . In some examples, methods include reducing or inhibiting one or more signs or symptoms associated with a tumor (such as hepatocellular carcinoma, glioblastoma, liver cancer, lung cancer, colon cancer, brain cancer, diabetes, or an inflammatory disease) modulated by GPCRs such as cannabinoid receptors (such as GPR55), beta adrenergic receptors, and serotonergic receptors

In some examples, the tumor is a primary tumor, such as a primary brain tumor expressing or regulated by GPCRs such as cannabinoid receptors (such as GPR55), beta adrenergic receptors, and serotonergic receptors. In some examples, the tumor is a glioblastoma or hepatocellular carcinoma expressing beta adrenergic receptors, and serotonergic receptors. In some examples, the tumor is a glioblastoma or hepatocellular carcinoma expressing, GPCRs such as GPR55 and serotonergic receptors but not expressing β2-AR. In some examples, the tumor is a glioblastoma or hepatocellular carcinoma expressing both GPR55, and β2-AR.

Disclosed methods include administering compound, such as NT015, NT016 or a combination thereof (and, optionally, one or more other pharmaceutical agents) depending upon the receptor population of the tumor, to a subject in a pharmaceutically acceptable carrier and in an amount effective to treat the tumor expressing a β2-AR, cannabinoid receptor, serotonergic receptor or a susceptible receptor or combination thereof, such as a primary tumor. Treatment of a tumor includes preventing or reducing signs or symptoms associated with the presence of such tumor (for example, by reducing the size or volume of the tumor or a metastasis thereof). Such reduced growth can in some examples decrease or slow metastasis of the tumor, or reduce the size or volume of the tumor by at least 10%, at least 20%, at least 50%, or at least 75%, such as between 10%-90%, 20%-80%, 30% 70%, 40%-60%, including a 10%, 15%, 20%, 25%, 30%, 35%, 40%, 45%, 50%, 55%, 60%, 65%, 70%, 75%, 80%, 90%, or 95% reduction. In another example, treatment includes reducing the invasive activity of the tumor in the subject, for example by reducing the ability of the tumor to metastasize. In some examples, treatment using the methods disclosed herein prolongs the time of survival of the subject.

Routes of administration useful in the disclosed methods include but are not limited to oral and parenteral routes, such as intravenous (IV), intraperitoneal (IP), rectal, topical, ophthalmic, nasal, and transdermal as described in detail above.

An effective amount of compound, such as NT015, NT016 or combination thereof will depend, at least, on the particular method of use, the subject being treated, the severity of the tumor, and the manner of administration of the therapeutic composition. A “therapeutically effective amount” of a composition is a quantity of a specified compound sufficient to achieve a desired effect in a subject being treated. For example, this may be the amount of NT015, NT016 or a combination thereof necessary to prevent or inhibit tumor growth and/or one or more symptoms associated with the tumor in a subject. Ideally, a therapeutically effective amount of a disclosed compound is an amount sufficient to prevent or inhibit a tumor, such as a brain or liver tumor growth and/or one or more symptoms associated with the tumor in a subject without causing a substantial cytotoxic effect on host cells.

Therapeutically effective doses of a disclosed compound or pharmaceutical composition can be determined by one of skill in the art, with a goal of achieving concentrations that are at least as high as the IC₅₀ of the applicable compound disclosed in the examples herein. An example of a dosage range is from about 0.001 to about 10 mg/kg body weight orally in single or divided doses. In particular examples, a dosage range is from about 0.005 to about 5 mg/kg body weight orally in single or divided doses (assuming an average body weight of approximately 70 kg; values adjusted accordingly for persons weighing more or less than average). For oral administration, the compositions are, for example, provided in the form of a tablet containing from about 1.0 to about 50 mg of the active ingredient, particularly about 2.5 mg, about 5 mg, about 10 mg, or about 50 mg of the active ingredient for the symptomatic adjustment of the dosage to the subject being treated. In one exemplary oral dosage regimen, a tablet containing from about 1 mg to about 50 mg active ingredient is administered two to four times a day, such as two times, three times or four times.

In other examples, a suitable dose for parental administration is about 1 milligram per kilogram (mg/kg) to about 100 mg/kg, such as a dose of about 10 mg/kg to about 80 mg/kg, such including about 1 mg/kg, about 2 mg/kg, about 5 mg/kg, about 20 mg/kg, about 30 mg/kg, about 40 mg/kg, about 50 mg/kg, about 80 mg/kg or about 100 mg/kg administered parenterally. However, other higher or lower dosages also could be used, such as from about 0.001 mg/kg to about 1 g/kg, such as about 0.1 to about 500 mg/kg, including about 0.5 mg/kg to about 200 mg/kg.

Single or multiple administrations of the composition comprising one or more of the disclosed compositions can be carried out with dose levels and pattern being selected by the treating physician. Generally, multiple doses are administered. In a particular example, the composition is administered parenterally once per day. However, the composition can be administered twice per day, three times per day, four times per day, six times per day, every other day, twice a week, weekly, or monthly. Treatment will typically continue for at least a month, more often for two or three months, sometimes for six months or a year, and may even continue indefinitely, i.e., chronically. Repeat courses of treatment are also possible.

The specific dose level and frequency of dosage for any particular subject may be varied and will depend upon a variety of factors, including the activity of the specific compound, the metabolic stability and length of action of that compound, the age, body weight, general health, sex and diet of the subject, mode and time of administration, rate of excretion, drug combination, and severity of the condition of the subject undergoing therapy.

### Selecting a Subject

Subjects can be screened prior to initiating the disclosed therapies, for example to select a subject in need of or at risk of developing a disorder or disease regulated by susceptible activity or expression. Briefly, the method can include screening subjects to determine if they have or are at risk of developing a GPCR regulated disease, such as if the subject is in need of tumor inhibition. Subjects having a tumor that expresses a susceptible receptor, such as GPR55, or is regulated by susceptible activity, such as a primary tumor, including a primary brain tumor, such as a glioblastoma, hepatocellular carcinoma, liver cancer, lung cancer, or colon cancer or at risk of developing such a tumor are selected. In one example, subjects are diagnosed with the tumor by clinical signs, laboratory tests, or both. For example, a tumor, such as a primary brain tumor, can be diagnosed by characteristic clinical signs, such as headaches, vomiting, seizures, dizziness, weight loss and various associated complaints. Diagnosis is generally by imaging analysis such as by magnetic resonance imaging (MRI) and confirmed by histology. In some examples, a subject is selected that does not have a bleeding disorder, such as an intracerebral hemorrhage.

In an example, a subject in need of the disclosed therapies is selected by detecting a tumor expressing a GPCR or regulated by its activity, such as by detecting susceptible activity or expression in a sample obtained from a subject identified as having, suspected of having or at risk of acquiring such a tumor. For example, detection of altered, such as at least a 10% alteration, including a 10%-90%, 20%-80%, 30%-70%, 40%-60%, such as a 10%, 15%, 20%, 25%, 30%, 35%, 40%, 45%, 50%, 55%, 60%, 65%, 70%, 75%, 80%, 90%, 95% alteration or more in susceptible expression or activity as compared to susceptible expression or activity in the absence of a primary tumor, indicates that the tumor can be treated using the compositions and methods provided herein which are susceptible regulators. In other examples, a subject is selected by detecting a primary brain tumor such as an astrocytoma or glioblastoma by MRI or positron emission tomography (PET) in a subject, or by checking for response of a tumor specimen from the tumor, circulation or other bodily fluid, by exposing the tumor sample to the disclosed compound and finding a response.

In some examples, a subject is selected by determining the subject has or is at risk of developing a disorder or disease, such as a tumor and/or cancer, which does not respond to (32-AR stimulation.

Pre-screening is not required prior to administration of the therapeutic agents disclosed herein (such as those including NT015, NT016 or a combination thereof).

### Exemplary Tumors

Exemplary tumors include tumors that express a susceptible receptor, such as GPR55, or serotonergic receptors or regulated by such, including primary tumors, such as a primary brain tumor. A primary brain tumor includes astrocytomas, glioblastomas, ependymoma, oligodendrogliomas, and mixed gliomas. Additional possible types of tumors associated with susceptible activity or expression include hematological tumors, such as leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of possible solid tumors which may express a susceptible receptor or be regulated by susceptible activity, include sarcomas and carcinomas, such as fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, liver cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma). In several examples, a tumor is a brain cancer, liver cancer, or lung cancer that expresses a susceptible receptor, such as GPR55. Tumors expressing a susceptible receptor, such as GPR55, can be identified by routine methods known to those of skill in the art including Western blot and histological studies with antibodies capable of detecting a susceptible receptor, such as GPR55

### Assessment

Following the administration of one or more therapies, subjects having a disorder or disease susceptible to disclosed compound/s or regulated by susceptible receptor, such as a tumor-expressing GPR55 (for example, a primary tumor) can be monitored for decreases in tumor growth, tumor volume or in one or more clinical symptoms associated with the tumor. In particular examples, subjects are analyzed one or more times, starting 7 days following treatment. Subjects can be monitored using any method known in the art including those described herein including imaging analysis.

### Additional Treatments and Additional Therapeutic Agents

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least a 20%, at least a 30%, at least a 40%, at least a 50%, or at least a 70% reduction in one or more signs or symptoms associated with the disorder or disease, such as a tumor susceptible to disclosed compound/s and/or regulated by susceptible receptor, including tumor size or volume.

In some examples, the method further includes administering a therapeutic effective amount of NT015, NT016 or a combination thereof with additional therapeutic treatments. In particular examples, prior to, during, or following administration of a therapeutic amount of an agent that prevents or inhibits a tumor regulated by a susceptible receptor, the subject can receive one or more other therapies. In one example, the subject receives one or more treatments to remove or reduce the tumor prior to administration of a therapeutic amount of a composition including NT015, NT016, or combination thereof.

Examples of such therapies include, but are not limited to, surgical treatment for removal or reduction of the tumor (such as surgical resection, cryotherapy, or chemoembolization), as well as anti-tumor pharmaceutical treatments which can include radiotherapeutic agents, anti-neoplastic chemotherapeutic agents, antibiotics, alkylating agents and antioxidants, kinase inhibitors, and other agents. Particular examples of additional therapeutic agents that can be used include microtubule-binding agents, DNA intercalators or cross-linkers, DNA synthesis inhibitors, DNA and/or RNA transcription inhibitors, antibodies, enzymes, enzyme inhibitors, and gene regulators. These agents (which are administered at a therapeutically effective amount) and treatments can be used alone or in combination. Methods and therapeutic dosages of such agents are known to those skilled in the art, and can be determined by a skilled clinician.

“Microtubule-binding agent” refers to an agent that interacts with tubulin to stabilize or destabilize microtubule formation thereby inhibiting cell division. Examples of microtubule - binding agents that can be used in conjunction with the disclosed therapy include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used and are known to those of ordinary skill in the art. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

The following classes of compounds are of use in the methods disclosed herein: Suitable DNA and/or RNA transcription regulators, including, without limitation, actinomycin D, daunorubicin, doxorubicin and derivatives and analogs thereof also are suitable for use in combination with the disclosed therapies. DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof. DNA synthesis inhibitors suitable for use as therapeutic agents include, without limitation, methotrexate, 5-fluoro-5′-deoxyuridine, 5-fluorouracil and analogs thereof. Examples of suitable enzyme inhibitors include, without limitation, camptothecin, etoposide, formestane, trichostatin and derivatives and analogs thereof. Examples of alkylating agents include carmustine or lomustine. Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof. Kinase inhibitors include Gleevac, Iressa, and Tarceva that prevent phosphorylation and activation of growth factors.

Other therapeutic agents, for example anti-tumor agents, that may or may not fall under one or more of the classifications above, also are suitable for administration in combination with the disclosed therapies. By way of example, such agents include adriamycin, apigenin, rapamycin, zebularine, cimetidine, and derivatives and analogues thereof.

In one example, at least a portion of the tumor (such as the primary brain tumor) is surgically removed (for example via cryotherapy), irradiated, chemically treated (for example via chemoembolization) or combinations thereof, prior to administration of the disclosed therapies (such as administration of NT015, NT016 or a combination thereof). For example, a subject having a primary brain tumor associated with susceptible receptor activity can have at least a portion of the tumor surgically excised prior to administration of the disclosed therapies. In an example, one or more chemotherapeutic agents are administered following treatment with a composition including NT015, NT016, or a combination thereof. In another particular example, the subject has a primary brain tumor and is administered radiation therapy, chemoembolization therapy, or both concurrently with the administration of the disclosed therapies.

### Additional Disorders and Diseases

As discussed above, in addition to methods of treating disease/ disorder susceptible to disclosed compound/s and / or susceptible regulator-regulated tumors, it is contemplated that the disclosed compounds possessing susceptible receptor modulatory activity, such as modulator of GPR55 activity, can be used to treat other conditions associated with susceptible receptor regulation, such as metabolic disorders and disease (e.g., obesity and diabetes), or inflammatory and neuropathic pain disorders, diseases associated with aging such as Alzheimer's, bone loss, muscle wasting (sarcopenia), osteoarthritis and loss of appetite, central nervous system conditions such as depression and anxiety and other diseases and disorders associated with susceptible receptor regulation.

Routes of administration useful in the disclosed methods include but are not limited to oral and parenteral routes, such as intravenous (IV), intraperitoneal (IP), rectal, topical, ophthalmic, nasal, and transdermal as described in detail above.

An effective amount of a disclosed compound or combination thereof will depend, at least, on the particular method of use, the subject being treated, the severity of the disorder/disease, and the manner of administration of the therapeutic composition. A “therapeutically effective amount” of a composition is a quantity of a specified compound sufficient to achieve a desired effect in a subject being treated. Ideally, a therapeutically effective amount of a disclosed compound is an amount sufficient to prevent or inhibit one or more symptoms associated with the particular disorder/disease in a subject without causing a substantial cytotoxic effect on host cells.

Therapeutically effective doses of a disclosed compound or pharmaceutical composition can be determined by one of skill in the art, with a goal of achieving concentrations that are at least as high as the IC₅₀ of the applicable compound disclosed in the examples herein. An example of a dosage range is from about 0.001 to about 10 mg/kg body weight orally in single or divided doses. In particular examples, a dosage range is from about 0.005 to about 5 mg/kg body weight orally in single or divided doses (assuming an average body weight of approximately 70 kg; values adjusted accordingly for persons weighing more or less than average). For oral administration, the compositions are, for example, provided in the form of a tablet containing from about 1.0 to about 50 mg of the active ingredient, particularly about 2.5 mg, about 5 mg, about 10 mg, or about 50 mg of the active ingredient for the symptomatic adjustment of the dosage to the subject being treated. In one exemplary oral dosage regimen, a tablet containing from about 1 mg to about 50 mg active ingredient is administered once to four times a day, such as one time, two times, three times or four times.

In other examples, a suitable dose for parental administration is about 1 milligram per kilogram (mg/kg) to about 100 mg/kg, such as a dose of about 10 mg/kg to about 80 mg/kg, such including about 1 mg/kg, about 2 mg/kg, about 5 mg/kg, about 20 mg/kg, about 30 mg/kg, about 40 mg/kg, about 50 mg/kg, about 80 mg/kg or about 100 mg/kg administered parenterally. However, other higher or lower dosages also could be used, such as from about 0.001 mg/kg to about 1 g/kg, such as about 0.1 to about 500 mg/kg, including about 0.5 mg/kg to about 200 mg/kg.

Single or multiple administrations of the composition comprising one or more of the disclosed compositions can be carried out with dose levels and pattern being selected by the treating physician. Generally, multiple doses are administered. In a particular example, the composition is parenterally administered once per day. However, the composition can be administered twice per day, three times per day, four times per day, six times per day, every other day, twice a week, weekly, or monthly. Treatment will typically continue for at least one month, more often for two or three months, sometimes for six months or a year, and may even continue indefinitely, that is, chronically. Repeat courses of treatment are also possible.

The specific dose level and frequency of dosage for any particular subject may be varied and will depend upon a variety of factors, including the activity of the specific compound, the metabolic stability and length of action of that compound, the age, body weight, general health, sex and diet of the subject, mode and time of administration, rate of excretion, drug combination, and severity of the condition of the subject undergoing therapy. In some examples, one or more disclosed compound with susceptible receptor activity is orally administered to a subject daily to treat one or more symptoms associated with an aging disorder or disease (such as Alzheimer's, sarcopenia, bone loss, or combinations thereof) or a central nervous system disorder or disease (such as anxiety or depression).

Subjects can be screened prior to initiating the disclosed therapies, for example to select a subject in need of or at risk of developing a disorder or disease susceptible to disclosed compound/s and/or regulated by susceptible receptor activity or expression. Briefly, the method can include screening subjects to determine if they have or are at risk of developing a susceptible receptor-regulated disorder or disease. Subjects having a disorder or disease that expresses a susceptible receptor, such as GPR55, or is regulated by susceptible receptor activity and/ or bearing a cancer susceptible to disclosed compound/s are selected. In one example, subjects are diagnosed by clinical signs, laboratory tests, or both known to those of ordinary skill in the art or disclosed herein (or both).

Pre-screening is not required prior to administration of the therapeutic agents disclosed herein (such as those including compound or a combination thereof).

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least a 20%, at least a 30%, at least a 40%, at least a 50%, or at least a 70% reduction in one or more signs or symptoms associated with the disorder or disease.

In some examples, the method further includes administering a therapeutic effective amount of one or more compound with additional therapeutic treatments. In particular examples, prior to, during, or following administration of a therapeutic amount of an agent that prevents or inhibits a tumor regulated by susceptible activity, the subject can receive one or more other therapies. In one example, the subject receives one or more treatments to remove or reduce one or more signs or symptoms associated with the susceptible receptor regulated disorder/disease prior to administration of a therapeutic amount of a composition including one or more compounds.

The subject matter of the present disclosure is further illustrated by the following non-limiting Examples.

### EXAMPLES

### Example 1

### Material and Methods

Synthesis of NT016—Dimethylhydroxylamine hydrochloride (29.6 mmol) with 4.79 gms of 1.1′-carbonyldiimidazole and 2.98 gms of trimethylamine were mixed in THF. The reaction mix was worked up on ice. The reaction was extracted with ethanol and dried over sodium sulfate. It was evaporated under reduced pressure to give 5.98 gms of yellow oil (yield 97%) of N,O-dimethyl-N-(3-(naphthalen-1-yl)prop-1-en-2-yl)hydroxylamine. This product (MW 229) was mixed with 18.2 ml of methyl magnesium chloride (3M in THF) with 30 ml of THF and stirred overnight below 0 C. The reaction was quenched by brine (50 ml) and pH was adjusted to 4 by addition of 1N HCl. The brine was washed by adding ethanol (200 ml) followed by drying over sodium sulfate. Chromatography with ethanol:hexane:1:10 was used to purify 4.2 g of yellow oil with a yield of 1-naphthalene acetic acid 84.5% over two steps. To 184 mg (10 mmol) of 1-naphthalene acetic acid (MW 186;26.88 mmol) and Tyramine (137 mg, 1 mmol) dissolved in dichloromethane (10 ml) acetic acid (120 mg, 20 mmol) was added. The mixture was stirred at RT for 30 minutes and then NaBH(OAc)₃ 424 mg was added dropwise. The resulting mixture was stirred at RT overnight. Chromatography (chloroform; methanol:20:1 was done to give 210 mg of white foam. This was tested by NMR. The molecular weight and purity was tested by mass spectrometry. The results of the NMR are as follows: HNMR (CDCl3, 300 MHz): 1.29 (dd, 3H, J1=15 Hz, J2=6 Hz), 2.90-3.31 (m, 5H), 3.53-3.57 (m, 1H), 3.84-3.90 (m, 1H), 6.72 (s, 4H), 7.32-7.34 (m, 2H), 7.47-7.53 (m, 2H), 7.52 (d, 1H, J=3 Hz), 7.83 (d, 1H, J=9 Hz), 8.14 (d, 1H, J=6 Hz).

Eagle's Minimum Essential Medium (E-MEM), trypsin solution, phosphate-buffered saline (PBS), fetal bovine serum (FBS), 100× solutions of sodium pyruvate (100 mM), L-glutamine (200 mM), and penicillin/streptomycin (a mixture of 10,000 units/ml penicillin and 10,000 gg/ml streptomycin) were obtained from Quality Biological (Gaithersburg, Md.).

Maintenance and Treatment of Cell Lines. Human HepG2 hepatocarcinoma cells and human U87MG glioma cells (ATCC, Manassas, Va.) were maintained in EMEM medium supplemented with 10% FBS (Hyclone, Logan, Utah). The human 1321N1 astrocytoma cells (European Collection of Cell Cultures, Sigma-Aldrich) were cultured in Dulbecco's modified Eagle's medium supplemented with 10% FBS and 2 mM L-glutamine. Human prostate carcinoma cell line PC3 was cultured in F-12K medium (Life Technologies) supplemented 10% FBS. Human lung carcinoma cell line A549 was cultured in DMEM+2 mM glutamine+10% FBS. ER positive, estrogen independent for growth MCF7/LCC9 cell line was cultured in IMEM (Biofluids) supplemented with 5% charcoal stripped calf serum. Medulloblastoma cell line DAOY was cultured in Eagle's MEM supplemented with 10% FBS. All cell lines except MDA-MB-321 were cultured at 37° C. in 5% CO2, and the medium was replaced every 2-3 days. MDA-MB-231 was grown in Leibovitz's L-15 medium with 10% FBS without CO2 supplementation.

Cell death assay. The extent of cell death and toxicity of the compounds was tested by XTT assay. The cells were plated in their respective media in 96-well plates. The compounds and were added in dilutions ranging from 0.01 nM to 200 μM and for control DMSO to 1% was used. After addition of test compounds the cells were incubated usually 24 or 48 hours or when cells showed a response by visual inspection under the microscope. For XTT assay first the medium in the wells was replaced with 200 μs of fresh phenol-red free medium. Then 50 μls of XTT solution with PMS was added to the wells. The XTT solution was made adding 0.02% phenazine methosulfate (Sigma-Aldrich #P9625) to 1 mg/ml XTT sodium salt made up in phenol-red free RPMI (Sigma-Aldrich #X4636). The plate was incubated for 4 hours at 37° C. and then the absorbance was read at a wavelength of 490 nM.

Caspase Assay. Caspase assay was conducted by using the Promega Caspase-Glo 3/7 kit. 100 ul of caspase-Glo 3/7 reagent was added to each well of a white walled 96-well plate containing 100 ul of blank, negative control cells or treated cells in culture medium. The contents of the wells were mixed gently and incubated at room temperature for 30 minutes to 3 hours. The luminescence of each sample was read in a luminometer.

Apoptosis Assay. The degree of apoptosis induced by drug treatment was assayed by flow cytometry using the Alexa Fluor® 488 annexin V/Dead Cell Apoptosis Kit (Invitrogen) following the standard manufacturer's protocol. Briefly, HepG2 cells (5×10⁵) were grown on 100 mm dishes for 24 hours followed by treatment with vehicle, compound, all in serum-free medium. Cells were subsequently harvested after a 24-hour incubation, washed in cold PBS, and resuspended in 100 μL of 1× annexin-binding buffer to maintain a density ˜1×10⁶ cells/mL, after which 5 μL Alexa Fluor® 488 annexin V and 1 μL 100 μg/mL propidium iodide were added to the cell suspensions. Cells were then incubated at room temperature for 15 minutes and 400 μL 1× annexin-binding buffer was added followed by gentle mixing. Stained cells were analyzed on a BDFACSCanto II flow cytometer.

Zebrafish xenograft assay. Zebrafish embryos with implanted A549 cells were imaged within 6 hours of cell injection (day 0) and treated with compounds. At 4 days post injection (day 4) the embryos were imaged again. The area of fluorescence due to the labeled cells was measured using Image J at day 0 (area 0) and day 4 (area 4). The change in area was calculated by subtracting area 0 from area 4 (=d area). D area was then normalized for the size of the initial tumor by dividing d area by area 0 (=d area/area 0).

Statistical Analysis. Results were expressed as relative to the control value. Studies were performed in at least two to three different culture preparations, and two to three dishes for each test condition were plated in each preparation. Results are expressed as means ±S.E. Student's t-test was used to make statistical comparisons between groups. Analyses were performed using the 5 SigmaPlot Software (Systat Software, Inc. San Jose, Calif.), Graphpad Prism 4 (GraphPad Software, Inc., La Jolla, Calif.) and Microsoft® Office Excel, 2003 (Microsoft Corp., Redmond, A), with p values≤0.05 considered significant.

### Example 2

This example demonstrates that NT015 and NT016 are potent inhibitors of brain, liver, breast, and lung cancer cell growth.

The effect of NT015 and NT016 on the growth of a variety of tumor cells was evaluated. An IC₅₀ value of less than 100 μM was considered active. As illustrated in Table 1 below, NT015 and NT016 were potent inhibitors of liver cancer cell growth, lung cancer cell growth, breast cancer cell growth, prostate cancer cell growth, CNS cancer cell growth.

These studies indicate that NT015 and NT016 are capable of inhibiting additional types of cancer growth, including liver, lung and breast cancer. One of skill in the art will appreciate that they also provide support for using analogs of these compounds, to reduce tumor growth, including treating cancer, such as liver, lung and cancer, in additional subjects, including humans.

TABLE 1
Effect of NT015, NT016 and related
compounds on cancer cells.

| | | | 1321N* | HepG2* | | MDA- | | | |
|---|---|---|---|---|---|---|---|---|---|
| | CID | | (brain | (liver | | MB- | | LCC- | |
| CID | number | Structure | cancer) | cancer) | PC3 | 231 | A549 | 9 | DAOY |
| NT001 | 3083544 | 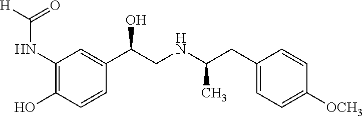 [SMILES: COc1ccc(C[C@@H](C)NC[C@H](O)c2ccc(O)c(NC=O)c2)cc1] | — | — | | | | | |
| | | 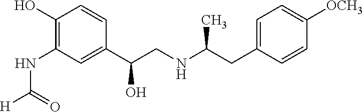 [SMILES: COc1ccc(C[C@H](C)NC[C@@H](O)c2ccc(O)c(NC=O)c2)cc1] | | | | | | | |
| NT002 | 2083 | 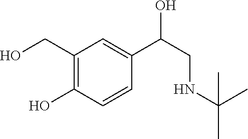 [SMILES: CC(C)(C)NCC(O)c1ccc(O)c(CO)c1] | — | — | | | | | |
| NT003 | 370 | 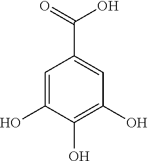 [SMILES: O=C(O)c1cc(O)c(O)c(O)c1] | 200 uM | 200 uM | | | | | |
| NT004 | 41393 | 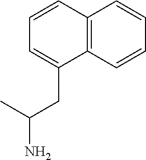 | 100 μM | 100 μM | | | | | |
| NT005 | | 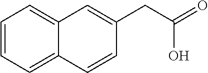 [SMILES: O=C(O)Cc1ccc2ccccc2c1] | — | — | | | | | |
| NT006 | 6862 | 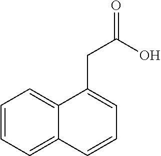 [SMILES: O=C(O)Cc1cccc2ccccc12] | — | — | | | | | |
| NT007 | 24849454 | 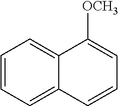 [SMILES: COc1cccc2ccccc12] | — | — | | | | | |
| NT008 | 156391 | 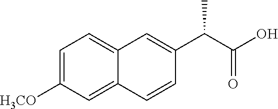 [SMILES: COc1ccc2cc([C@H](C)C(=O)O)ccc2c1] | — | — | | | | | |
| NT009 | 98089 | 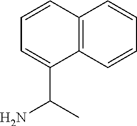 | 100 μM | 100 μM | | | | | |
| NT010 | 3857145 | 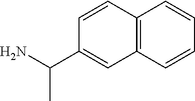 | 200 μM | 200 μM | | | | | |
| NT011 | 10584226 | 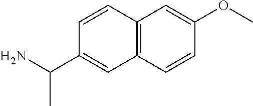 | 200 μM | 200 μM | | | | | |
| NT012 | CD5006568 | 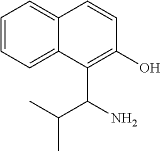 [SMILES: CC(C)C(N)c1c(O)ccc2ccccc12] | — | — | | | | | |
| NT013 | | 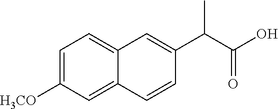 [SMILES: COc1ccc2cc(C(C)C(=O)O)ccc2c1] | — | — | | | | | |
| NT014 | | 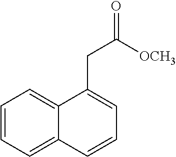 [SMILES: COC(=O)Cc1cccc2ccccc12] | — | — | | | | | |
| NT015 | 10219723 | 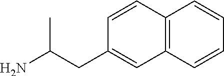 | 200 μM | 200 μM | 200 uM | — | # | # | # |
| NT016 | | 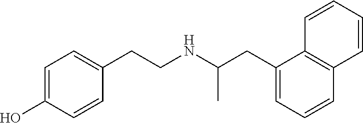 [SMILES: CC(Cc1cccc2ccccc12)NCCc1ccc(O)cc1] | 100 uM | 100 uM | 200 uM | — | 100 uM | 100 uM | 100 uM |
| NT017 | | 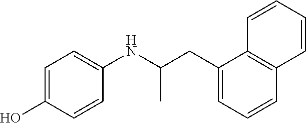 [SMILES: CC(Cc1cccc2ccccc12)Nc1ccc(O)cc1] | 100 uM | No effect | 100 uM | — | 100 uM | — | 100 uM |
| NT018 | | 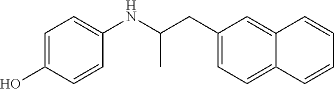 [SMILES: CC(Cc1ccc2ccccc2c1)Nc1ccc(O)cc1] | 100 uM | 200 uM | 100 uM | — | 100 uM | — | 100 uM |
| NT019 | | 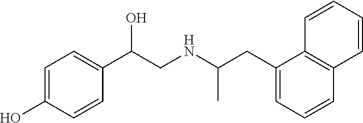 [SMILES: CC(Cc1cccc2ccccc12)NCC(O)c1ccc(O)cc1] | 100 uM | 100 uM | 100 uM | — | 100 uM | — | 100 uM |
| NT020 | | 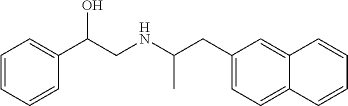 [SMILES: CC(Cc1ccc2ccccc2c1)NCC(O)c1ccccc1] | 100 uM | 100 uM | 100 uM | 100 uM | 100 uM | 100 uM | 100 uM |
| NT021 | | 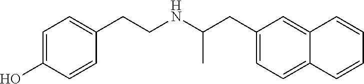 [SMILES: CC(Cc1ccc2ccccc2c1)NCCc1ccc(O)cc1] | 100 uM | 200 uM | 200 uM | 100 uM | 100 uM | 100 uM | 100 uM |
| NT022 | | 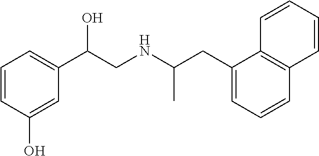 [SMILES: CC(Cc1cccc2ccccc12)NCC(O)c1cccc(O)c1] | 100 uM | 100 uM | 100 uM | 100 uM | 100 uM | 100 uM | 100 uM |
| NT023 | | 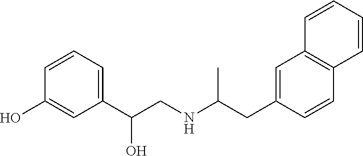 [SMILES: CC(Cc1ccc2ccccc2c1)NCC(O)c1cccc(O)c1] | 100 uM | 100 uM | 100 uM | 100 uM | 100 uM | 100 uM | 100 uM |
| NT024 | | 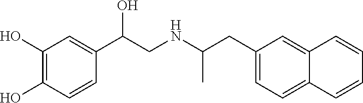 [SMILES: CC(Cc1ccc2ccccc2c1)NCC(O)c1ccc(O)c(O)c1] | — | — | — | — | — | — | — |
| NT025 | | 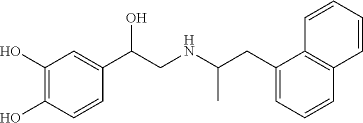 [SMILES: CC(Cc1cccc2ccccc12)NCC(O)c1ccc(O)c(O)c1] | — | — | — | — | — | — | — |
| NT026 | | 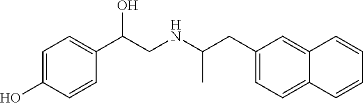 [SMILES: CC(Cc1ccc2ccccc2c1)NCC(O)c1ccc(O)cc1] | 100 uM | 100 uM | 100 uM | 100 uM | 100 uM | 100 uM | 100 uM |
| NT027 | | 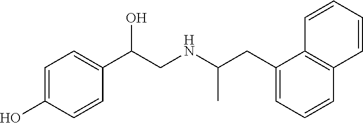 [SMILES: CC(Cc1cccc2ccccc12)NCC(O)c1ccc(O)cc1] | 100 uM | 100 uM | 100 uM | 100 uM | 100 uM | 100 uM | 100 uM |
| NT029 | | 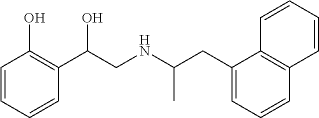 [SMILES: CC(Cc1cccc2ccccc12)NCC(O)c1ccccc1O] | 100 uM | 100 uM | 200 uM | —# | 100 uM | 100 uM | 100 uM |
| NT030 | | 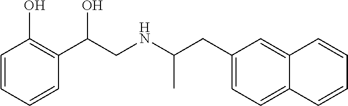 [SMILES: CC(Cc1ccc2ccccc2c1)NCC(O)c1ccccc1O] | 100 uM | 100 uM | 100 uM | 100 uM | 100 uM | 100 uM | 100 uM |
| NT031 | 4581 | 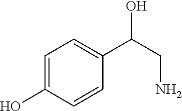 [SMILES: NCC(O)c1ccc(O)cc1] | — | — | NT | — | — | — | — |
| NT032 | 24891195 | 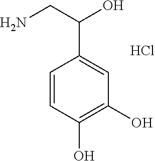 [SMILES: NCC(O)c1ccc(O)c(O)c1 ; Cl] | — | — | NT | — | — | — | — |
| NT033 | 24847145 | 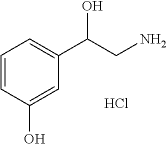 [SMILES: NCC(O)c1cccc(O)c1 ; Cl] | — | — | NT | — | — | — | — |
| NT034 | | 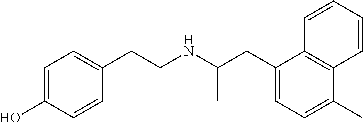 [SMILES: Cc1ccc(CC(C)NCCc2ccc(O)cc2)c2ccccc12] | 200 uM | 200 uM | —# | 100 uM | —# | 100 uM | |
| NT035 | | 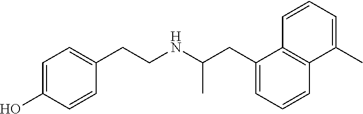 [SMILES: Cc1cccc2c(CC(C)NCCc3ccc(O)cc3)cccc12] | 100 uM | 100 uM | 100 uM | 100 uM | 100 uM | 100 uM | 100 uM |
| NT036 | | 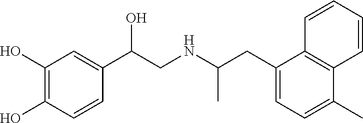 [SMILES: Cc1ccc(CC(C)NCC(O)c2ccc(O)c(O)c2)c2ccccc12] | — | NT | NT | — | — | — | |
| NT037 | | 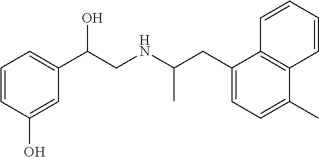 [SMILES: Cc1ccc(CC(C)NCC(O)c2cccc(O)c2)c2ccccc12] | 100 uM | NT | NT | 100 uM | 100 uM | 100 uM | 100 uM |
| NT038 | | 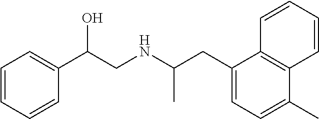 [SMILES: Cc1ccc(CC(C)NCC(O)c2ccccc2)c2ccccc12] | NT | NT | NT | 100 uM | 100 uM | 100 uM | 100 uM |
| NT039 | | 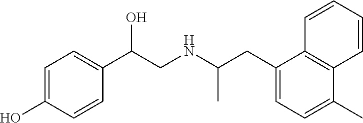 [SMILES: Cc1ccc(CC(C)NCC(O)c2ccc(O)cc2)c2ccccc12] | NT | NT | NT | — | 100 uM | 100 uM | 100 uM |
| NT040 | | 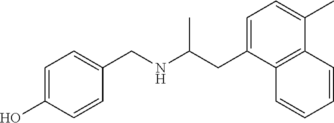 [SMILES: Cc1ccc(CC(C)NCc2ccc(O)cc2)c2ccccc12] | NT | NT | NT | NT | NT | NT | NT |
| NT041 | | 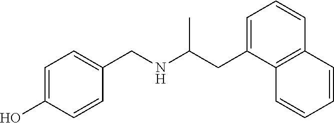 [SMILES: CC(Cc1cccc2ccccc12)NCc1ccc(O)cc1] | NT | NT | NT | NT | NT | NT | NT |
| NT042 | | 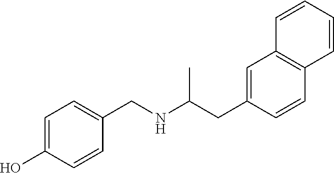 [SMILES: CC(Cc1ccc2ccccc2c1)NCc1ccc(O)cc1] | NT | NT | NT | NT | NT | NT | NT |

*Greater than 50% inhibition.
#200 uM not tested.
NT—Not tested.

### Example 3

### Treatment of a tumor responsive to NT015 and NT016

This example describes a method that can be used to treat a tumor in a human subject by administration of a composition comprising NT015, NT016 analogue or a combination thereof at a therapeutically effective amount to reduce or inhibit on or more signs or symptoms associated with the tumor, such as a glioblastoma or hepatocellular carcinoma. Although particular methods, dosages, and modes of administrations are provided, one skilled in the art will appreciate that variations can be made without substantially affecting the treatment.

A subject with a glioblastoma or hepatocellular carcinoma is selected based upon clinical symptoms. The composition including the desired compounds is intraperitoneally administered to the subject at a concentration of 30 mg/kg/day for the first 10 days and 50 mg/kg/day for the remaining 32 days. Tumor growth is then assessed 7 days, 14 days, 21 days, 30 days, and 42 days following treatment. In one example, the effectiveness of the treatment is determined by imaging methods, including non-invasive, high-resolution modalities, such as computed tomography (CT) and especially magnetic resonance imaging (MRI). For example, contrast agent uptake is monitored to determine the effectiveness of the treatment. A decrease in permeability to the blood-brain barrier marked by an at least twenty percent (20%) decrease in uptake of a contrast agent as compared to reference value or that measured prior to treatment indicates the treatment is effective. Also, a twenty-percent (20%) reduction in tumor size as compared to tumor size prior to treatment is considered to be an effective treatment. In some examples, a subject is administered an intravenous formulation of NT015, or NT016 2 Kg formulation. In some examples, a subject is administered an intravenous formulation of NT015 or NT016 at a concentration ranging from 0.1 to 10 mg/kg for 4 days as a single agent or in combination with other standard agents used in cancer chemotherapy over a two week period as a continuous or pulsed therapy. In some examples, a subject is administered orally a 25 mg/kg dose of NT015 or NT016 formulated as a single agent or as a combination on a daily basis for a certain period of time, such as 1 month, 2 months, 3 months, 4 months, 5 months, 6 months followed by additional periods if desired, based upon regression of or inhibition of tumor growth.

### Example 4

### Use of Disclosed Compositions Including NT015, NT016 (or Both) as an Adjuvant Therapy

This example describes a method that can be used to reduce, prevent, or retard tumor growth in a human subject that has been treated for a malignant astrocytoma.

A subject with an astrocytoma is selected based upon clinical symptoms. The primary form of treatment of the malignant astrocytoma is open surgery. For subjects that are not surgical candidates, either radiation or chemotherapy is used as the initial treatment. Following the initial treatment, a subject is administered a pharmaceutical composition containing NT015 or NT016 orally daily for an indefinite period of time. The reoccurrence of tumor growth is monitored by imaging methods, including non-invasive, high-resolution modalities, such as CT and MRI.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

What is claimed is:

1. A compound, or a pharmaceutically acceptable salt thereof, having the structure of Formula I: 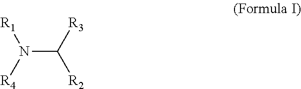
wherein,
R₁ and R₃ are either hydrogen or alkyl groups;
R₄ is hydrogen, substituted or unsubstituted alkyl, substituted or unsubstituted aryl, or substituted or unsubstituted alkyl chains terminating in a substituted or unsubstituted aryl;
R₂ is a substituted or unsubstituted naphthyl group optionally linked by a substituted or unsubstituted alkyl chain; 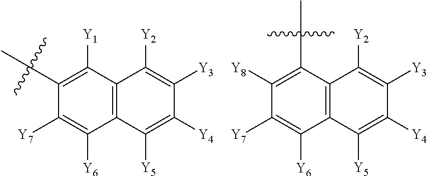
wherein,
each Y₁-Y₈ are independently selected from hydrogen, deuterium, halogen (F, Cl, Br, I), hydroxyl, alkoxy, nitrogenous substituents such as primary amines, substituted secondary and tertiary amines, sulfurous substituents including SH, sulfoxides, sulfones, sulfonamides, substituted or unsubstituted alkyl and substituted or unsubstituted aryls.

2. The compound of claim 1, wherein
R₁ and R₃ are either hydrogen or alkyl groups;
R₄ is hydrogen, substituted or unsubstituted alkyl, substituted or unsubstituted aryl, or substituted or unsubstituted alkyl chains terminating in a substituted or unsubstituted aryls;
R₂ is a substituted or unsubstituted naphthyl group optionally linked by a substituted or unsubstituted alkyl chain; and
each Y₁-Y₈ are independently selected from hydrogen, deuterium, halogen (F, Cl, Br, I), hydroxyl, alkoxy, primary amines, substituted secondary and tertiary amines, thiols, sulfoxides, sulfones, sulfanamides, substituted or unsubstituted alkyl and substituted or unsubstituted aryls.

3. The compound of claim 1, wherein
R₁ and R₃ are either hydrogen or alkyl groups;
R₄ is hydrogen, substituted or unsubstituted aryl or substituted or unsubstituted alkyl chains terminating in a substituted or unsubstituted aryls;
R₂ is a substituted or unsubstituted naphthyl group optionally linked by a substituted or unsubstituted alkyl chain; and
each Y₁-Y₈ are independently selected from hydrogen, deuterium, halogen (F, Cl, Br, I), hydroxyl, alkoxy, primary amines, substituted secondary and tertiary amines, thiols, sulfoxides, sulfones, sulfanamides, substituted or unsubstituted alkyl and substituted or unsubstituted aryls.

4. The compound of claim 1, wherein
R₁ and R₃ are either hydrogen or alkyl groups;
R₄ is hydrogen or a substituted or unsubstituted alkyl chains terminating in a substituted or unsubstituted aryls;
R₂ is a substituted or unsubstituted naphthyl group optionally linked by a substituted or unsubstituted alkyl chain; and
each Y₁-Y₈ are independently selected from hydrogen, deuterium, halogen (F, Cl, Br, I), hydroxyl, alkoxy, primary amines, substituted secondary and tertiary amines, thiols, sulfoxides, sulfones, sulfanamides, substituted or unsubstituted alkyl and substituted or unsubstituted aryls.

5. The compound of claim 1, wherein
R₁ and R₃ are either hydrogen or alkyl groups;
R₄ is hydrogen or an alkyl chain terminating in a hydroxyl substituted aryls;
R₂ is a substituted or unsubstituted naphthyl group optionally linked by a substituted or unsubstituted alkyl chain; and
each Y₁-Y₈ are independently selected from hydrogen, deuterium, halogen (F, Cl, Br, I), hydroxyl, alkoxy, primary amines, substituted secondary and tertiary amines, thiols, sulfoxides, sulfones, sulfanamides, substituted or unsubstituted alkyl and substituted or unsubstituted aryls.

6. The compound of claim 1, wherein
R₁ and R₃ are either hydrogen or alkyl groups;
R₄ is hydrogen or an alkyl chain terminating in a hydroxyl substituted aryls;
R₂ is a substituted or unsubstituted naphthyl group optionally linked by a substituted or unsubstituted alkyl chain; and
each Y₁-Y₈ are independently selected from hydrogen, hydroxyl, and alkoxy substituents.

7. The compound of any of claims 1-6, wherein the compound is selected from the group consisting of: 1-naphthalen-1-yl)ethan-1-amine; 1-(naphthalen-2-yl)ethan-1-amine; 1-(6-methoxynaphthalen-2-yl)ethan-1-amine; 1-(1-amino-2-methylpropyl)naphthalen-2-ol; 1-(naphthalen-2-yl)propan-2-amine; 4-(2-((1-(naphthalen-1-yl)propan-2-yl)amino)ethyl)phenol; 4-((1-(naphthalen-1-yl)propan-2-yl)amino)phenol; 4-((1-(naphthalen-2-yl)propan-2-yl)amino)phenol; 4-(1-hydroxy-2-((1-(naphthalen-1-yl)propan-2-yl)amino)ethyl)phenol; 2-((1-(naphthalen-2-yl)propan-2-yl)amino)-1-phenylethan-1-ol; 4-(2-((1-(naphthalen-2-yl)propan-2-yl)amino)ethyl)phenol; 3-(1-hydroxy-2-((1-(naphthalen-1-yl)propan-2-yl)amino)ethyl)phenol; 3-(1-hydroxy-2-((1-(naphthalen-2-yl)propan-2-yl)amino)ethyl)phenol; 4-(1-hydroxy-2-((1-(naphthalen-2-yl)propan-2-yl)amino)ethyl)benzene-1,2-diol; 4-(1-hydroxy-2-((1-(naphthalen-1-yl)propan-2-yl)amino)ethyl)benzene-1,2-diol; 4-(1-hydroxy-2-((1-(naphthalen-2-yl)propan-2-yl)amino)ethyl)phenol; 4-(1-hydroxy-2-((1-(naphthalen-1-yl)propan-2-yl)amino)ethyl)phenol; 2-(1-hydroxy-2-((1-(naphthalen-1-yl)propan-2-yl)amino)ethyl)phenol; 2-(1-hydroxy-2-((1-(naphthalen-2-yl)propan-2-yl)amino)ethyl)phenol; 4-(2-((1-(4-methylnaphthalen-1-yl)propan-2-yl)amino)ethyl)phenol; 4-(2-((1-(5-methylnaphthalen-1-yl)propan-2-yl)amino)ethyl)phenol; 4-(1-hydroxy-2-((1-(4-methylnaphthalen-1-yl)propan-2-yl)amino)ethyl)benzene-1,2-diol; 3-(1-hydroxy-2-((1-(4-methylnaphthalen-1-yl)propan-2-yl)amino)ethyl)phenol; 2-((1-(4-methylnaphthalen-1-yl)propan-2-yl)amino)-1-phenylethan-1-ol; 4-(1-hydroxy-2-((1-(4-methylnaphthalen-1-yl)propan-2-yl)amino)ethyl)phenol; 4-(((1-(4-methylnaphthalen-1-yl)propan-2-yl)amino)methyl)phenol; 4-(((1-(naphthalen-1-yl)propan-2-yl)amino)methyl)phenol and 4-(((1-(naphthalen-2-yl)propan-2-yl)amino)methyl)phenol.

8. The compound of any one of claims 1-6, wherein the compound is 4-(2-((1-(naphthalen-1-yl)propan-2-yl)amino)ethyl)phenol.

9. A method of treating cancer or other disorder/disease susceptible to treatment by using the compound of any one of claims 1-8.

10. A method of administering a therapeutically effective amount of the compound of any one of claims 1-8.

11. A method of administering a therapeutically effective amount of a compound, wherein the compound is selected from the group consisting of: 1-(naphthalen-1-yl)propan-2-amine; 1-naphthalen-1-yl)ethan-1-amine; 1-(naphthalen-2-yl)ethan-1-amine; 1-(6-methoxynaphthalen-2-yl)ethan-1-amine; 1-(1-amino-2-methylpropyl)naphthalen-2-ol; 1-(naphthalen-2-yl)propan-2-amine; 4-(2-((1-(naphthalen-1-yl)propan-2-yl)amino)ethyl)phenol; 4-((1-(naphthalen-1-yl)propan-2-yl)amino)phenol; 4-((1-(naphthalen-2-yl)propan-2-yl)amino)phenol; 4-(1-hydroxy-2-((1-(naphthalen-1-yl)propan-2-yl)amino)ethyl)phenol; 2-((1-(naphthalen-2-yl)propan-2-yl)amino)-1-phenylethan-1-ol; 4-(2-((1-(naphthalen-2-yl)propan-2-yl)amino)ethyl)phenol; 3-(1-hydroxy-2-((1-(naphthalen-1-yl)propan-2-yl)amino)ethyl)phenol; 3-(1-hydroxy-2-((1-(naphthalen-2-yl)propan-2-yl)amino)ethyl)phenol; 4-(1-hydroxy-2-((1-(naphthalen-2-yl)propan-2-yl)amino)ethyl)benzene-1,2-diol; 4-(1-hydroxy-2-((1-(naphthalen-1-yl)propan-2-yl)amino)ethyl)benzene-1,2-diol; 4-(1-hydroxy-2-((1-(naphthalen-2-yl)propan-2-yl)amino)ethyl)phenol; 4-(1-hydroxy-2-((1-(naphthalen-1-yl)propan-2-yl)amino)ethyl)phenol; 2-(1-hydroxy-2-((1-(naphthalen-1-yl)propan-2-yl)amino)ethyl)phenol; 2-(1-hydroxy-2-((1-(naphthalen-2-yl)propan-2-yl)amino)ethyl)phenol; 4-(2-((1-(4-methylnaphthalen-1-yl)propan-2-yl)amino)ethyl)phenol; 4-(2-((1-(5-methylnaphthalen-1-yl)propan-2-yl)amino)ethyl)phenol; 4-(1-hydroxy-2-((1-(4-methylnaphthalen-1-yl)propan-2-yl)amino)ethyl)benzene-1,2-diol; 3-(1-hydroxy-2-((1-(4-methylnaphthalen-1-yl)propan-2-yl)amino)ethyl)phenol; 2-((1-(4-methylnaphthalen-1-yl)propan-2-yl)amino)-1-phenylethan-1-ol; 4-(1-hydroxy-2-((1-(4-methylnaphthalen-1-yl)propan-2-yl)amino)ethyl)phenol; 4-(((1-(4-methylnaphthalen-1-yl)propan-2-yl)amino)methyl)phenol; 4-(((1-(naphthalen-1-yl)propan-2-yl)amino)methyl)phenol and 4-(((1-(naphthalen-2-yl)propan-2-yl)amino)methyl)phenol.

12. A method of administering a therapeutically effective amount of 1-(naphthalen-2-yl)propan-2-amine or 4-(2-((1-(naphthalen-1-yl)propan-2-yl)amino)ethyl)phenol.

13. A method of administering a therapeutically effective amount of a compound, wherein the compound is selected from the group consisting of: 1-(naphthalen-1-yl)propan-2-amine; 1-naphthalen-1-yl)ethan-1-amine; 1-(naphthalen-2-yl)ethan-1-amine; 1-(6-methoxynaphthalen-2-yl)ethan-1-amine; 1-(1-amino-2-methylpropyl)naphthalen-2-ol; 1-(naphthalen-2-yl)propan-2-amine; 4-(2-((1-(naphthalen-1-yl)propan-2-yl)amino)ethyl)phenol; 4-((1-(naphthalen-1-yl)propan-2-yl)amino)phenol; 4-((1-(naphthalen-2-yl)propan-2-yl)amino)phenol; 4-(1-hydroxy-2-((1-(naphthalen-1-yl)propan-2-yl)amino)ethyl)phenol; 2-((1-(naphthalen-2-yl)propan-2-yl)amino)-1-phenylethan-1-ol; 4-(2-((1-(naphthalen-2-yl)propan-2-yl)amino)ethyl)phenol; 3-(1-hydroxy-2-((1-(naphthalen-1-yl)propan-2-yl)amino)ethyl)phenol; 3-(1-hydroxy-2-((1-(naphthalen-2-yl)propan-2-yl)amino)ethyl)phenol; 4-(1-hydroxy-2-((1-(naphthalen-2-yl)propan-2-yl)amino)ethyl)benzene-1,2-diol; 4-(1-hydroxy-2-((1-(naphthalen-1-yl)propan-2-yl)amino)ethyl)benzene-1,2-diol; 4-(1-hydroxy-2-((1-(naphthalen-2-yl)propan-2-yl)amino)ethyl)phenol; 4-(1-hydroxy-2-((1-(naphthalen-1-yl)propan-2-yl)amino)ethyl)phenol; 2-(1-hydroxy-2-((1-(naphthalen-1-yl)propan-2-yl)amino)ethyl)phenol; 2-(1-hydroxy-2-((1-(naphthalen-2-yl)propan-2-yl)amino)ethyl)phenol; 4-(2-((1-(4-methylnaphthalen-1-yl)propan-2-yl)amino)ethyl)phenol; 4-(2-((1-(5-methylnaphthalen-1-yl)propan-2-yl)amino)ethyl)phenol; 4-(1-hydroxy-2-((1-(4-methylnaphthalen-1-yl)propan-2-yl)amino)ethyl)benzene-1,2-diol; 3-(1-hydroxy-2-((1-(4-methylnaphthalen-1-yl)propan-2-yl)amino)ethyl)phenol; 2-((1-(4-methylnaphthalen-1-yl)propan-2-yl)amino)-1-phenylethan-1-ol; 4-(1-hydroxy-2-((1-(4-methylnaphthalen-1-yl)propan-2-yl)amino)ethyl)phenol; 4-(((1-(4-methylnaphthalen-1-yl)propan-2-yl)amino)methyl)phenol; 4-(((1-(naphthalen-1-yl)propan-2-yl)amino)methyl)phenol and 4-(((1-(naphthalen-2-yl)propan-2-yl)amino)methyl)phenol or a combination thereof.

14. A method of administering a therapeutically effective amount of 1-(naphthalen-2-yl)propan-2-amine or 4-(2-((1-(naphthalen-1-yl)propan-2-yl)amino)ethyl)phenol or a combination thereof.

15. The method of any one of claims 9-14 wherein the disorder or disease is liver cancer, brain cancer, lung cancer, breast cancer or any cancer that responds to the compound of claim 1.

16. The method of any one of claims 9-14 wherein the disorder or disease is liver cancer, brain cancer, lung cancer or breast cancer.

17. The method of claim 16 wherein the disorder or disease is liver cancer.

18. The method of claim 16 wherein the disorder or disease is brain cancer.

19. The method of claim 16 wherein the disorder or disease is lung cancer.

20. The method of claim 16 wherein the disorder or disease is breast cancer.

21. The method of any one of claims 9-14, wherein inhibiting one or more signs or symptoms associated with the disease or disorder comprises inhibiting cellular growth, such as tumor or cancer cell growth (or both), tumor volume, or a combination thereof.

22. The method of any one of claims 9-16, further comprising administering an additional therapeutic agent, such as prior to, concurrent with, or subsequent to administering a compound.

23. The method of claim 22, wherein the additional therapeutic agent is a chemotherapeutic agent or agent with antitumor activity.

24. The method of any one of claims 10-23, wherein administering a therapeutically effective amount of a compound is done so with use of a pharmaceutically acceptable carrier.

25. The method of any one of claims 9-24, wherein the subject is a human.
